(19) United States
(12) Patent Application Publication
(10) Pub. No.: US 2006/0088475 A1
(43) Pub. Date: Apr. 27, 2006

(54) Self-immolative magnetic resonance imaging contrast agents sensitive to beta-glucuronidase

(75) Inventors: Joseph A. Duimstra, Santa Cruz, CA (US); Thomas J. Meade, Wilmette, IL (US)

Correspondence Address: MEDLEN & CARROLL, LLP;Suite 350, 101 Howard Street, San Francisco, CA, 94105, US

(73) Assignee: Northwestern University, Evanston, IL (US)

(21) Appl. No.: 11/125,985

(22) Filed: May 10, 2005

Related U.S. Application Data
(60) Provisional application No. 60/569,755, filed on May 10, 2004.

(51) Int. Cl. A61K 49/10

(52) U.S. Cl. 424/9.361

(57) ABSTRACT

The present invention relates to magnetic resonance imaging (MRI) contrast agent. In particular, the present invention provides MRI contrast agents that are sensitive to the enzyme beta-glucoronidase. The MRI contrast agents provide compositions and methods for non-invasive diagnostic imaging of tissues, including necrotic tumors.

The present invention claims priority to U.S. Provisional Patent Application Ser. No. 60/569,755, filed May 10, 2004, the disclosure of which is herein incorporated by reference in its entirety.

This invention was made with government support under Grant No. DAMD17-02-1 awarded by the Department of Defense. The Government may have certain rights in the invention.

### FIELD OF THE INVENTION

The present invention relates to magnetic resonance imaging (MRI) contrast agent. In particular, the present invention provides MRI contrast agents that are sensitive to the enzyme beta-glucoronidase. The MRI contrast agents provide compositions and methods for non-invasive diagnostic imaging of tissues, including necrotic tumors.

### BACKGROUND OF THE INVENTION

Magnetic resonance imaging (MRI) is a diagnostic and research procedure that uses high magnetic fields and radio-frequency signals to produce images. The most abundant molecular species in biological tissues is water. It is the quantum mechanical “spin” of the water proton nuclei that ultimately gives rise to the signal in all imaging experiments. In MRI the sample to be imaged is placed in a strong static magnetic field and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans.

MR images are typically displayed on a gray scale with black the lowest and white the highest measured intensity (I). This measured intensity I═C*M, where C is the concentration of spins (in this case, water concentration) and M is a measure of the magnetization present at time of the measurement. Although variations in water concentration (C) can give rise to contrast in MR images, it is the strong dependence of the rate of change of M on local environment that is the source of image intensity variation in MRI. Two characteristic relaxation times, T₁ and T₂, govern the rate at which the magnetization can be accurately measured. T₁ is the exponential time constant for the spins to decay back to equilibrium after being perturbed by the RF pulse. In order to increase the signal-to-noise ratio (SNR) a typical MR imaging scan (RF & gradient pulse sequence and data acquisition) is repeated at a constant rate for a predetermined number of times and the data averaged. The signal amplitude recorded for any given scan is proportional to the number of spins that have decayed back to equilibrium since the previous scan. Thus, regions with rapidly decaying spins (i.e. short T₁ values) will recover all of their signal amplitude between successive scans.

The measured intensities in the final image will accurately reflect the spin density (i.e. water content). Regions with long T₁ values compared to the time between scans will progressively lose signal until a steady state condition is reached and will appear as darker regions in the final image. Changes in T₂ (spin-spin relaxation time) result in changes in the signal linewidth (shorter T₂ values) yielding larger linewidths. In extreme situations the linewidth can be so large that the signal is indistinguishable from background noise. In clinical imaging, water relaxation characteristics vary from tissue to tissue, providing the contrast that allows the discrimination of tissue types. Moreover, the MRI experiment can be setup so that regions of the sample with short T₁ values and/or long T₂ values are preferentially enhanced so called T₁-weighted and T₂-weighted imaging protocol.

There is a rapidly growing body of literature demonstrating the clinical effectiveness of paramagnetic contrast agents used in MRI. The capacity to differentiate regions/tissues that may be magnetically similar but histologically distinct is a major impetus for the preparation of these agents. In the design of MRI agents, attention should be given to a variety of properties that will ultimately effect the physiological outcome apart from the ability to provide contrast enhancement. Two important properties that should be considered are biocompatability and proton relaxation enhancement. Biocompatability is influenced by several factors including toxicity, stability (thermodynamic and kinetic), pharmacokinetics and biodistribution. Proton relaxation enhancement (or relaxivity) is chiefly governed by the choice of metal and rotational correlation times.

In general, contrast agents are made potent by incorporating metals with unpaired d or f electrons. For example, T1 contrast agents often include a lanthanide metal ion, usually Gd³⁺, that is chelated to a low molecular-weight molecule in order to limit toxicity. T2-agents often consist of small particles of magnetite (FeO—Fe₂O₃) that are coated with dextran. Both types of agents interact with mobile water in tissue to produce contrast; the details of this microscopic interaction differ depending on the agent type. While existing contrast agents are useful in many circumstances, they are not able to image the full range of biological states of tissue that one would like to analyze.

Thus, a new generation of MRI contrast agents is required to adapt this powerful imaging technology to the needs of biological research and clinical diagnostic applications.

### SUMMARY OF THE INVENTION

The present invention provides compositions and methods involving magnetic resonance imaging (MRI) contrast agent. In particular, the present invention provides MRI contrast agents that are sensitive to the enzyme glucoronidase enzymes. The MRI contrast agents provide compositions and methods for non-invasive diagnostic imaging of tissues, including necrotic tumors.

For example, in some embodiments, the present invention provides a composition comprising a compound for use as a contrast agent in magnetic resonance imaging, said compound comprising: a sensor component and a an MRI agent (e.g., contained in a macrocycle), wherein the contrast agent is configured decompose and release the MRI agent in the presence of a glucuronidase (e.g., beta-glucuronidase). In preferred embodiments, the sensor component comprises beta-glucuronic acid. In yet other preferred embodiments, the compound further comprises a linker that attaches the sensor to the macrocycle. The present invention also provides kits containing such compositions. In some particularly preferred embodiments, the contrast agent is the structure shown in FIG. 1 or derivatives thereof.

The present invention also provides methods for imaging a tissue, the methods comprising the steps of a) exposing a tissue to a contrast agent comprising a sensor component and an MRI agent, wherein said contrast agent is configured to decompose and release the MRI agent in the presence of a glucuronidase; and imaging the tissue via magnetic resonance imaging (e.g., by detecting the MRI agent). In some preferred embodiments, the tissue comprises necrotic tumor tissue. Thus, in such embodiments, the method finds use for research and diagnostic identification and analysis of tumor tissue, response to drugs or other therapies, and the like. The invention may be used for any tissue, including tissue located in vivo in a subject (e.g., a human subject).

### DESCRIPTION OF THE DRAWINGS

FIG. 1 depicts a macrocycle containing an MRI agent of the present invention.

FIGS. 2A-2C shows a route of synthesis of the macrocycle depicted in FIG. 1.

FIG. 3 shows synthesis scheme 1 and the compounds GdHP-DO3A, EGad and EGadMe.

FIG. 4 shows synthesis scheme 2.

FIG. 5 shows synthesis scheme 3.

FIG. 6 shows synthesis scheme 4A and 4B.

FIG. 7 shows synthesis scheme 5.

FIG. 8 shows synthesis scheme 6.

FIG. 9 depicts T₁ relaxivity of GdHPDO3A, 1, and 2 at 60 MHz, 37° C., pH=7.4. α-10 mM MOPS, 100 mM NaCl. b-100 mM sodium phosphate. c-100 mM sodium phosphate, 0.01% (w/v) bovine serum albumin (BSA). Error is ±1 S.D. of duplicate measurements.

FIG. 10 depicts T₁ Relaxivity of 1 and 2 at 60 MHz, 37° C., pH=7.4. α-100 mM sodium phosphate, 0.01% (w/v) BSA, 24 mM NaHCO₃. b-10 mM MOPS, 24 mM NaHCO₃. c-100 mM NaCl, 0.9 mM Na₂HPO₄, 30 mM NaHCO₃, 0.13 mM sodium citrate, 2.3 mM sodium lactate. d-Male human blood serum. Error is ±1 S.D. of duplicate measurements.

FIG. 11 depicts representative kinetics of enzyme catalyzed hydrolysis of 1 monitored by UV-visible (20 second sampling rate) at 37° C. ▴: 0.2 mM 1, 1.0 mg/ml β-glucuronidase, 100 mM sodium phosphate, 0.01% (w/v) bovine serum albumin (BSA), 24 mM NaHCO3, pH=7.4, λ=422 nm. ♦: 0.2 mM 1, 0.1 mg/ml β-glucuronidase, 100 mM sodium acetate, pH=5.0 at 37° C., λ=354 nm.

FIG. 12 depicts kinetics of enzyme catalyzed hydrolysis of 1 monitored by bulk water T1 relaxation (60 MHz, 37° C.). Error bars on data (filled symbols) are ±1 S.D. of 3 independent measurements. Open symbols are control runs without enzyme. ▪: 0.2 mM 1, 1.0 mg/ml β-glucuronidase, 100 mM sodium phosphate, 0.01% (w/v) bovine serum albumin (BSA), pH=7.4. ▴: 0.2 mM 1, 1.0 mg/ml β-glucuronidase, 100 mM sodium phosphate, 0.01% (w/v) bovine serum albumin (BSA), 24 mM NaHCO3, pH=7.4. ♦: 0.2 mM 1, 0.1 mg/ml β-glucuronidase, 100 mM sodium acetate, pH=5.0. ●: 0.2 mM 1, 1.0 mg/ml β-glucuronidase, male human blood serum.

### DEFINITIONS

To facilitate understanding of the invention, a number of terms are defined below.

As used herein, the term “magnetic resonance imaging (MRI) device” or “MRI” incorporates all devices capable of magnetic resonance imaging or equivalents. The methods of the invention can be practiced using any such device, or variation of a magnetic resonance imaging (MRI) device or equivalent, or in conjunction with any known MRI methodology. For example, in magnetic resonance methods and apparatuses, a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. Magentic field gradients are applied to spatially encode the signals. The resulting signals are detected by radio-frequency coils placed adjacent to the tissue or area of the body of interest. See, e.g., U.S. Pat. Nos. 6,144,202; 6,128,522; 6,127,775; 6,119,032; 6,111,410; 5,555,251; 5,455,512; 5,450,010, each of which is herein incorporated by reference in its entirety. MRI and supporting devices are manufactured by, e.g., Bruker Medical GMBH; Caprius; Esoate Biomedica; Fonar; GE Medical Systems (GEMS); Hitachi Medical Systems America; Intermagnetics General Corporation; Lunar Corporation; MagneVu; Marconi Medicals; Philips Medical Systems; Shimadzu; Siemens; Toshiba America Medical Systems; and Varian; including imaging systems, by, e.g., Silicon Graphics.

As used herein, the term “sample” is used in its broadest sense. In one sense it can refer to a tissue sample. In another sense, it is meant to include a specimen or culture obtained from any source, as well as biological. In another sense, it is meant to include inanimate objects such as non-living items. In another sense, it is meant to include whole living systems (including humans).

As used herein, the term “biological entity” is used in its broadest sense. A biological entity may be obtained from animals (including humans) and encompass fluids, solids, organs, whole bodies, internal cavities, tissues, and gases. Biological samples include, but are not limited to whole organs, such as a brain, heart, lung, and the like; blood products, such as plasma, serum and the like; tissue products, such as skin, vulnerable plaque in carotid arteries, and the like. These examples are not to be construed as limiting the sample types applicable to the present invention.

As used herein, the terms “processor,” “imaging software,” “software package,” or other similar terms are used in their broadest sense. In one sense, the terms “processor,” “imaging software,” “software package,” or other similar terms refer to a device and/or system capable of obtaining, processing, and/or viewing images obtained with an imaging device.

As used herein, the terms “paramagnetic metal ion”, “paramagnetic ion” or “metal ion” refer to a metal ion that is magnetized parallel or antiparallel to a magnetic field to an extent proportional to the field. Generally, these are metal ions that have unpaired electrons. Examples of suitable paramagnetic metal ions, include, but are not limited to, gadolinium III (Gd+3 or Gd(III)), iron III (Fe+3 or Fe(III)), manganese II (Mnt2 or Mn(II)), yttrium III (Yt+3 or Yt(III)), dysprosium (Dy+3 or Dy(III)), and chromium (Cr(III) or Cr+3). In a preferred embodiment the paramagnetic ion is the lanthanide atom Gd(III), due to its high magnetic moment (u²=63 BM2), a symmetric electronic ground state (S8), and its current approval for diagnostic use in humans.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass) but for whom the stage, location, or form of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds.

### DETAILED DESCRIPTION OF THE INVENTION

The present invention provides Magnetic Resonance Imaging (MRI) Contrast Agents (CA) that are sensitive to the enzyme beta-glucuronidase. These contrast agents are based upon the change in the longitudinal relaxation time (T₁) of the hydrogen protons of bulk water molecules in the presence of a paramagnetic ion. The contrast agents of the present invention find use in any imaging application of a tissue or other sample that has can be differentiated by the amount of beta-glucuronidase associated with the tissue or sample. For example, beta-glucuronidase is present in high extracellular levels near necrotic tumors due to an immune response (Bosslet et al., Cancer Res., 58, 1195, 1998). The invention thus provides a non-invasive diagnostic for necrotic tumors by modulating the CA's access to water molecules. This capability provided by the present invention accomplishes the desired result by a different mechanism than the only other known MRI agent sensitive to beta-glucuronidase (J.-L. Guerquin-Kern, NMR Biomed., 13, 306, 2000), which provides a compound that is detected by a shift in ¹⁹F resonance and is not as sensitive to enzyme concentration as the compositions and methods of the present invention.

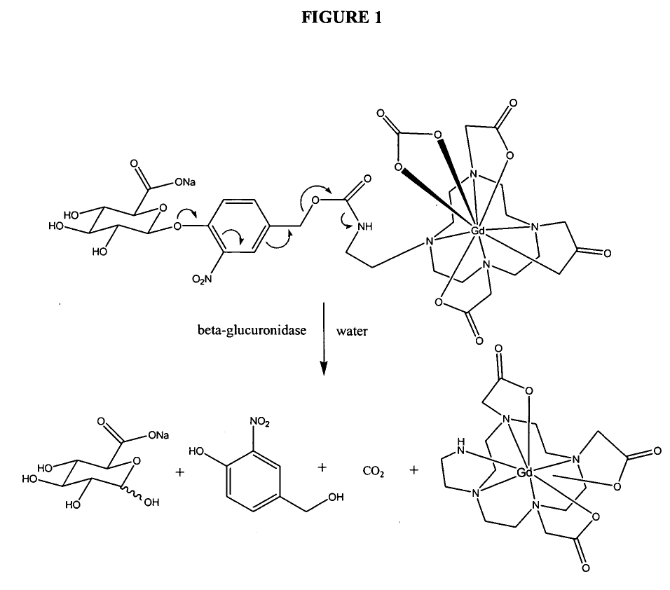

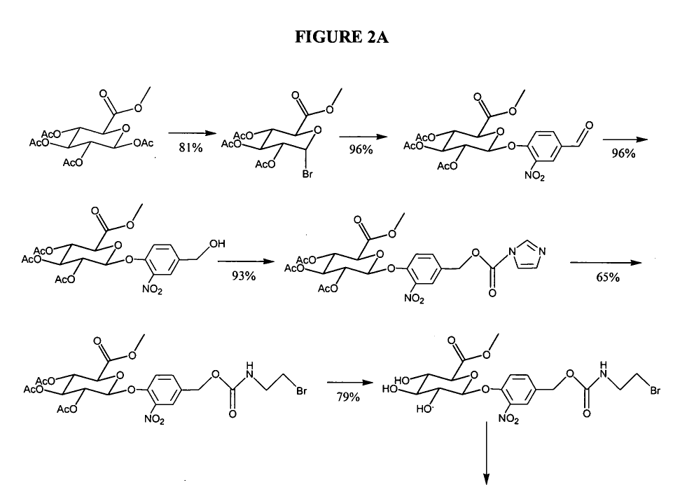

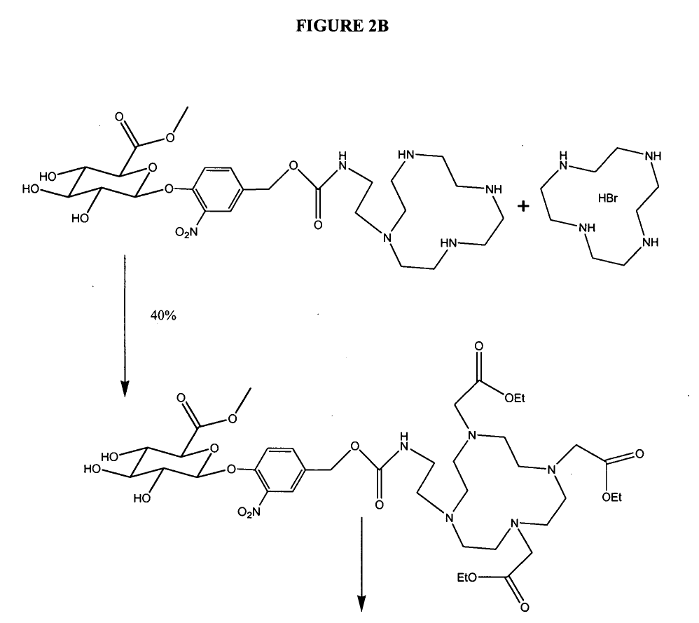

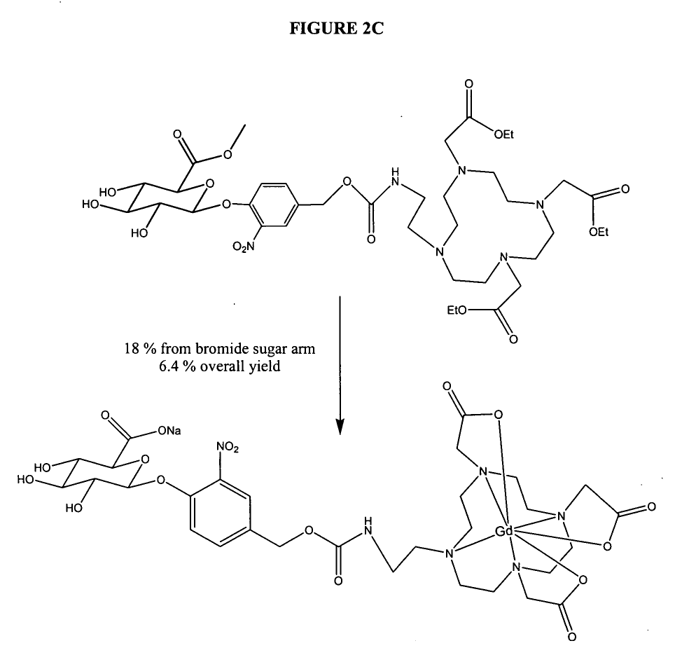

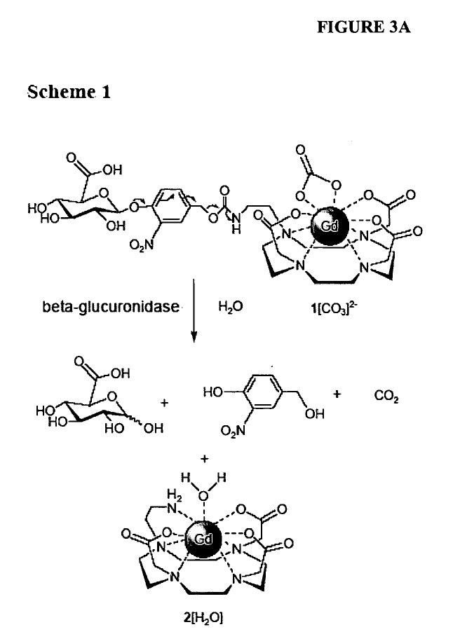

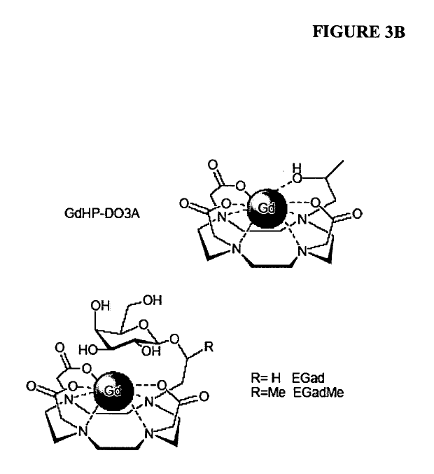

In preferred embodiments, the contrast agents of the present invention comprise three main parts. The first is the sensor. This is preferably a beta-glucuronic acid moiety. However, any component that is capable of reacting with beta-glucuronidase to cause a chemical change in the contrast agent so as to dissociate the sensor from an associated macrocycle containing an MRI agent may be used. The second is a linker that chemically associates the sensor to the macrocycle containing an MRI agent. The third is the macrocycle containing the MRI agent, preferably based on a gadolinium (III) ion (See, e.g., FIGS. 1 and 3). The mechanism of action of a preferred embodiment is based upon enzyme catalyzed hydrolysis of the glycosidic bond, followed by decomposition of the linker resulting in release of the MRI agent (FIGS. 1 and 3), although an understanding of the mechanism is not necessary to practice the present invention and the present invention is not limited to any particular mechanism of action. The contrast agent of the present invention is the first example of the use of a self-decomposable or immolative linker. This type of linker is known to be effective in delivery of chemotherapeutic prodrugs and has fast enzyme hydrolysis kinetics (J.-C. Florent, et al., J. Med. Chem., 41, 3572, 1998). Furthermore, the efficacy of the contrast agent is modulated by the extent of coordination of the pendant linker. The degree of coordination determines the number of water molecules directly bound to the gadolinium (III) center, which in turn is directly proportional to the spin-lattice relaxation time of bulk water. The synthesis of preferred agents of the present invention are depicted in FIGS. 2-8 and described in the Examples.

Thus, in some embodiments, the present invention provides a new class of q-modulated MR contrast agents that use a self-immolative mechanism for activation and detection. For example, in some embodiments the present invention provides a Gd(III) MR contrast agent whose effect on water proton T1 relaxation is modulated by hydrolysis of B-glucuronic acid (See, e.g., Examples 2-4, FIG. 3). In preferred embodiments, the agent possesses a self-immolative linker.

While preferred embodiments of the present invention is shown in FIGS. 1 and 3, the contrast agents of the present invention may be configured and used a wide variety of ways using components known in the art. For example, U.S. Pat. Nos. 6,770,261, 6,713,046, 6,713,045, 6,673,333, 6,656,450, 6,521,209, 6,232,295, 6,123,921, 5,980,862, 5,900,228, and 5,707,605, each of which is herein incorporated by reference in its entirety, describe a wide variety of configurations, compositions, and applications of contrast agents that may be adapted to the compositions and methods of the present invention.

A first feature to be considered during the design stage is the selection of the metal atom, which will dominate the measured relaxivity of the complex. Paramagnetic metal ions, as a result of their unpaired electrons, act as potent relaxation enhancement agents. They decrease the T₁ and T₂ relaxation times of nearby spins. Some paramagnetic ions decrease the T₁ without causing substantial linebroadening (e.g. gadolinium (III), (Gd³⁺)), while others induce drastic linebroadening (e.g. superparamagnetic iron oxide). The mechanism of T₁ relaxation is generally a through space dipole-dipole interaction between the unpaired electrons of the paramagnet (the metal atom with an unpaired electron) and bulk water molecules (water molecules that are not “bound” to the metal atom) that are in fast exchange with water molecules in the metal's inner coordination sphere (are bound to the metal atom).

Appropriate metal ions for use in the present invention include, but are not limited to, the transition, lanthanide and actinide elements. Preferably, the metal ion is selected from the group consisting of Gd(III), Mn(II), Cu(II), Cr(III), Fe(II), Fe(III), Co(II), Er(II), Ni(II), Eu(III) and Dy(III), with Gd(III) especially preferred.

Once the appropriate metal has been selected, a suitable ligand or chelate is found to render the complex nontoxic. Several factors influence the stability of chelate complexes include enthalpy and entropy effects (e.g. number, charge and basicity of coordinating groups, ligand field and conformational effects). Various molecular design features of the ligand can be directly correlated with physiological results. For example, the presence of a single methyl group on a given ligand structure can have a pronounced effect on clearance rate. To date, a number of chelators have been used, including diethylenetriaminepentaacetic (DTPA), 1,4,7,10-tetraazacyclododecane'-N,N′N″,N′″-tetracetic acid (DOTA), and derivatives thereof. See U.S. Pat. Nos. 5,155,215, 5,087,440, 5,219,553, 5,188,816, 4,885,363, 5,358,704, 5,262,532, and Meyer et al., Invest. Radiol. 25: S53 (1990), each of which is herein incorporated by reference in their entireties. Of course the primary criteria in selecting a chelator for use in the present invention is the ability of the chelator to coordinate a metal ion. In addition to DTPA and DOTA, Ethylenediaminetetraacetic acid (“EDTA”) and cyclic dietheylene triamine pentaacetic acid (“cDTPA”) find use with the present invention. A variety of other chelators are known in the art.

A wide variety of linkers may be used in the contrast agents of the present invention. Preferred linkers of the present invention are self-decomposable or immolative linkers in response to chemical modification of the sensor and/or linker by an enzyme that specifically modifies the sensor and/or linker. The linkers may also include groups to provide desired steric, solubility, and/or biocompatibility properties to the contrast agent. Preferred groups that may be used in the linker include, but are not limited to, alkyl and aryl groups, including substituted alkyl and aryl groups and heteroalkyl (particularly oxo groups) and heteroaryl groups, including alkyl amine groups, as defined above. Preferred groups include p-aminobenzyl, substituted p-aminobenzyl, diphenyl and substituted diphenyl, alkyl furan such as benzylfuran, carboxy, and straight chain alkyl groups of 1 to 10 carbons in length. Particularly preferred groups include p-aminobenzyl, methyl, ethyl, propyl, butyl, pentyl, hexyl, acetic acid, propionic acid, aminobutyl, p-alkyl phenols, 4-alkylimidazole, carbonyls, OH, COOH, glycols, etc.

The contrast agents of the present invention may further comprise one or more additional components that provide a desired functionality. For example, the compositions of the invention may optionally have at least one targeting moiety. In some embodiments, the targeting moiety replaces a coordination atom, although this is not generally preferred in clinical applications, as this may increase toxicity. By “targeting moiety” herein is meant a functional group which serves to target or direct the complex to a particular location, cell type, diseased tissue, or association. In general, the targeting moiety is directed against a target molecule. As will be appreciated by those in the art, the MRI contrast agents of the invention are generally injected intraveneously; thus preferred targeting moieties are those that allow concentration of the agents in a particular localization. In a preferred embodiment, the agent is partitioned to the location in a non-1:1 ratio. Thus, for example, antibodies, cell surface receptor ligands and hormones, lipids, sugars and dextrans, alcohols, bile acids, fatty acids, amino acids, peptides and nucleic acids may all be attached to localize or target the contrast agent to a particular site.

In a preferred embodiment, the targeting moiety allows targeting of the MRI agents of the invention to a particular tissue, the surface of a cell or a subcellular location. That is, in a preferred embodiment the MRI agents of the invention need not be taken up into the cytoplasm of a cell to be activated.

In a preferred embodiment, the targeting moiety is a peptide. For example, chemotactic peptides have been used to image tissue injury and inflammation, particularly by bacterial infection; see WO 97/14443, hereby expressly incorporated by reference in its entirety.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Hum(Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Imunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells.

In addition, antibodies against physiologically relevant carbohydrates may be used, including, but not limited to, antibodies against markers for breast cancer (CA15-3, CA549, CA27.29), mucin-like carcinoma associated antigen (MCA), ovarian cancer (CA125), pancreatic cancer (DE-PAN-2), and colorectal and pancreatic cancer (CA19, CA50, CA242).

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), estrogen receptor (estrogen); low density lipoprotein receptor, high density lipoprotein receptor, leptin receptor, interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-.alpha. and TGF-.beta.), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones above. Receptor ligands include ligands that bind to receptors such as cell surface receptors, which include hormones, lipids, proteins, glycoproteins, signal transducers, growth factors, cytokines, and others.

As will be appreciated by those in the art, the MRI compositions of the invention may take on a wide variety of different conformations, as outlined herein. In a preferred embodiment, the MRI agents are “monomers.” Alternatively, in a preferred embodiment, the MRI contrast agents of the invention comprise more than one metal ion, such that the signal is increased. In a preferred embodiment, the MRI agents of the invention comprise at least two paramagnetic metal ions, each with a chelator; that is, multimeric MRI agents are made. In a preferred embodiment, the chelators are linked together, either directly or through the use of a linker such as a coupling moiety or polymer. For example, using substitution groups that serve as functional groups for chemical attachment on the chelator, attachment to other chelators may be accomplished.

In one embodiment, the chelators are linked together directly, using at least one functional group on each chelator. In this embodiment, the chelators of the invention include one or more substitution groups that serve as functional groups for chemical attachment. Suitable functional groups include, but are not limited to, amines (preferably primary amines), carboxy groups, and thiols (including SPDP, alkyl and aryl halides, maleimides, .alpha.-haloacetyls, and pyridyl disulfides) are useful as functional groups that can allow attachment.

In some embodiments, it may be desirable to increase the blood clearance times (or half-life) of the MRI agents of the invention. This has been done, for example, by adding carbohydrate polymers, including polyethylene glycol, to the chelator (see U.S. Pat. Nos. 5,155,215 and 5,605,672). Thus, one embodiment utilizes polysaccharides as groups on the compositions of the invention.

A preferred embodiment utilizes complexes which cross the blood-brain barrier. Thus, as is known in the art, a DOTA derivative which has one of the carboxylic acids replaced by an alcohol to form a neutral DOTA derivative has been shown to cross the blood-brain barrier. Thus, for example, neutral complexes are designed that cross the blood-brain barrier.

The contrast agents of the present invention may also be co-administered with one or more additional imaging, diagnostic, or therapeutic agents.

The present invention provides methods of using the contrast agents. One embodiment of the present invention involves magnetic resonance-based imaging techniques. The magnetic resonance imaging techniques employed in the present invention are known and are described, for example, in Kean & Smith, (1986) Magnetic Resonance Imaging: Principles and Applications, Williams and Wilkins, Baltimore, Md. Standard equipment, conditions and techniques can be used to generate images; appropriate equipment, conditions and techniques can be determined in the course of experimental design. When in vivo MRI experiments are performed in the context of the present invention, they will be performed on a suitable device.

In embodiments of the present invention, a contrast enhancement agent can be introduced into a biological structure disposed in a subject. The mode of administration of a contrast enhancement agent of the invention to a sample or subject can determine the sites and/or cells in the organism to which an agent will be delivered. The contrast agents of the present invention will generally be administered in admixture with a pharmaceutical diluent selected with regard to the intended route of administration and standard pharmaceutical practice. The preparations can be injected into a subject parenterally, for example, intra-arterially or intravenously. For parenteral administration, a preparation can be used, e.g., in the form of a sterile, aqueous solution; such a solution can contain other solutes, including, but not limited to, salts or glucose in quantities that will make the solution isotonic. In another aspect, a contrast enhancement agent can be injected directly into a tumor.

When a contrast enhancement agent of the present invention is administered to humans, the prescribing physician will ultimately determine the appropriate dosage for a given human subject, and this can be expected to vary according to the weight, age and response of the individual as well as the nature and severity of the patient's condition.

Preferred embodiments of the present invention provide methods for imaging cancerous cells or tissues. For example, beta-glucuronidase has been implicated in breast, colorectal and small cell lung carcinomas. Beta-glucuronidase hydrolyzes the glucuronide bond at the non-reducing termini of glycosamino-carbohydrates. A variety of substrates are cleaved by beta-glucuronidase, including, but not limited to, phenolphthalein glucuronide, 5-bromo-4-chloro-3-indoly-β-glucuronide, etc.

The contrast agents of the present invention may be used in any method where a differential concentration of a target molecule that interacts with the sensor is to be imaged or analyzed. For example, the concentration of beta-glucuronidase has been shown to be low in well-differentiated cell lines and high in poorly differentiated (carcinoma) cell lines. In addition, beta-glucuronidase activity has been detected in stromal cells which penetrate tumors and in necrotic areas of solid tumors, where it is liberated by host inflammatory components, mainly by monocytes and granulocytes.

The present invention also provides kits comprising the contrast agents of the present invention. In preparing a kit of the present invention, in some embodiments, it is desirable to lyophilize the contrast enhancement agent in the same vial in which it will be distributed. An aqueous solution of the contrast enhancement agent herein disclosed is added to the vial after filtering through a sterilizing filtration system, such as a 0.22 micron filter typically used in sterilizing proteins or peptides. The contents of each vial can then be lyophilized and afterwards the vials capped and sealed under sterile conditions. A sterile final product is desirable when the product is going to be used for parenteral administration. In general the most useful container for use as a vial are the glass bottles typically used for lyophilizing biological materials. Another suitable container is a two-compartment syringe, wherein one compartment contains the lyophilized imaging agent cake and the other compartment contains the aqueous diluent. After lyophilization is complete, the vacuum within the vials or ampules can be released by filling the system with an inert gas, stoppered in place using standard equipment and then crimp sealed. Such a method will ensure a sterile final product.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); cm (centimeters); g (grams); 1 or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); and pmol (picomoles).

### Example 1

### Materials and Methods

General Methods: All reagents were used as purchased. 1,4,7,10-tetraazacyclododecane (cyclen) was obtained from Strem. Prohance was purified from the clinically available sample from Bracco Inc. using HPLC. Bovine liver β-glucuronidase [EC 3.2.1.31] Sigma cat G 0251 and BSA fraction V Sigma cat A 3059 and male human blood serum Sigma cat H 1388 were procured from Sigma. Dry solvents where indicated were obtained from Aldrich as anhydrous Sure-Seal bottles. Water was purified using a Millipore Milli-Q Synthesis purifier. Sugar-containing compounds were visualized on silica TLC plates with CAM stain (1 g (NH₄)₄CE(SO₄)₄, 2.5 g (NH₄)₄MO₂O₇, 6 ml conc. H₂SO₄, 94 ml water), while compounds containing unmetallated cyclen could be easily detected using a platinum stain (150 mg K₂PtCl₆, 10 ml 1 N HCl, 90 ml water, 3 g KI). NMR spectra were recorded on either a Varian Mercury 400 MHz or Varian Inova 500 MHz instrument. Peaks were referenced to an internal TMS standard. Infrared spectra were measured using a KBr plate on a Biorad FTS-60 FTIR spectrometer. Electrospray mass spectra were obtained via direct infusion of a methanolic solution of the compound of interest on a Varian 1200 L single quadrupole mass spectrometer. Elemental analysis was performed by Desert Analytics (Tucson, Ariz.). ICP-MS were recorded on a VG Elemental PQ Excell spectrometer standardized with eight concentrations spanning the range 0-50 ppb Gd(III). One ppb In(III) was used as the internal standard for all runs.

HPLC: LC-MS: Analytical LC-MS was performed on a computer controlled Varian Prostar system consisting of a 410 autosampler equipped with a 100 μL sample loop, two 210 pumps with 5 ml/min heads, a 363 fluorescence detector, a 330 photodiode array (PDA) detector, and a 1200 L single quadrupole ESI-MS. All runs were executed with a 0.8 ml/min flow rate using a ThermoElectron 4.6×150 mm 5 μm Aquasil C18 column, with a 3:1 split directing one part to the MS and 3 parts to the series-connected light detectors. Mobile phase consisted of water (solvent A) and HPLC-grade acetonitrile (solvent B) except where noted. All injections were full-loop.

Preparative LC: The preparative system is a Varian Prostar. Two 210 pumps with 25 ml/min heads fed a 5 ml manual inject sample loop. Detection was performed after a 20:1 split by a two channel 325 UV-visible detector and, on the low-flow leg, an HP 1046A fluorescence detector. The mobile phases were the same as in the LC-MS instrument. Preparative runs were typically 50-100 mg dissolved in water and run at 15 ml/min on a ThermoElectron 20×250 mm 5 μm Aquasil C18 column.

Methyl 1-(4-formyl-2-nitrophenyl)-2,3,4-tri-O-acetyl-β-D-glucopyronuronate (6) (See, e.g., Florent et al., J. Med. Chem. 1998, 41, 3572-3581): Methyl 1-bromo-2,3,4-tri-O-acetyl-α-D-glucopyronuronate (See, e.g., Bollenback et al., A. J. Am. Chem. Soc. 1955, 77, 3310-3315) (10.75 g, 27.1 mmol) was dissolved in 250 ml anhydrous MeCN. 4-hydroxy-3-nitrobenzaldehyde (7.64 g, 45.7 mmol) was then added followed by 28.5 g (123 mmol) of Ag₂O. The resulting slurry was stirred in the dark under N₂ for 4h. The solution was filtered through Celite to remove solids and the filtrate concentrated in vacuo. The residue was brought up in EtOAc (400 ml) and washed with saturated NaHCO₃ (6×200 ml), water and brine. The organic layer was dried over MgSO₄, filtered and concentrated in vacuo. The beige solid was triturated with hexanes yielding 6 (12.52 g, 96%). ¹H NMR (500 MHz, DMSO-d₆) δ 2.01, 2.02, 2.03 (3s, 3×3H, OAc), 3.64 (s, 3H, COOCH₃), 4.80 (d, 1H, H-5, J=10 Hz), 5.15 (m, 2H, H-2, H-4), 5.74 (m, 1H, H-3), 5.94 (d, 1H, H-1 J=8 Hz), 7.64 (d, 1H, ArH, J=9 Hz), 8.22 (dd, 1H, ArH, J=9 Hz, J′=1.5 Hz), 8.44 (d, 1H, ArH, J=1.5 Hz), 9.98 (s, 1H, CHO); C NMR (126 MHz, DMSO-d₆) δ20.17, 20.22, 20.26, 52.65, 68.49, 69.67, 70.43, 71.18, 97.25, 117.58, 126.23, 131.07, 134.74, 140.25, 152.10, 166.85, 168.72, 169.34, 169.49, 190.48; IR (KBr plate) v 2956, 1756, 1700, 1612, 1538, 1368, 1235, 1074, 1039 cm⁻¹;ESI-MSm/z(M+Na)⁺506.1; Anal. Calcd for C₂₀H₂₁NO₁₃: C 49.69; H 4.38; N 2.90; Found C, 49.92; H 4.55; N 2.80.

Methyl 1-(4-hydroxymethyl-2-nitrophenyl)-2,3,4-tri-O-acetyl-β-D-glucopyronuronate (7) (See, e.g., Florent et al., J. Med. Chem. 1998, 41, 3572-3581; Leu et al., J. Med. Chem. 1999, 42, 3623-3628): 1.41 g (37.3 mmol) NaBH₄ were added to a stirring solution of 12.03 g (24.9 mmol) 6 and 5 g silica gel at 0° C. in 300 ml 1:5 IPA:CHCl₃. After 45 min, the solution was poured into 300 ml ice water and filtered through Celite. The layers were separated and the organic fraction washed with brine, dried (MgSO₄), concentrated in vacuo, and triturated with Et₂O, yielding 7 as a white solid (11.65 g, 96%). ¹H NMR (500 MHz, DMSO-d₆) δ 1.99 (s, 3H, OAc), 2.02 (s, 6H, OAc), 3.33 (br OH), 3.64 (s, 3H, COOCH₃), 4.51 (d, 2H, CH₂OH, J=5.5 Hz), 4.73 (d, 1H, H-5, J=10 Hz), 5.10 (m, 2H, H-2, H-4), 5.44 (m, 1H, H-3), 5.71 (d, 1H, H-1 J=8 Hz), 7.38 (d, 1H, ArH, J=8 Hz), 7.62 (d, 1H, ArH, J=8 Hz), 7.80 (s, 1H, ArH); ¹³C NMR (126 MHz, DMSO-d₆) δ 20.19, 20.22, 20.28, 52.64, 61.32, 68.73, 69.94, 70.75, 71.02, 98.06, 117.70, 122.30, 131.97, 138.54, 140.18, 146.92, 166.92, 168.74, 169.32, 169.51; IR (KBr plate) v 3527, 1756, 1535, 1367, 1232, 1077, 1039 cm⁻¹; ESI-MS m/z (M+Na)⁺508.3; Anal. Calcd for C₂₀H₂₃NO₁₃: C 49.49; H 4.78; N 2.89; Found C 49.50; H 4.90; N 3.12.

Methyl 1-(4-(2-bromo-ethylcarbamoyloxymethyl)-2-nitrophenyl)-2,3,4-tri-O-acetyl-β-D-glucopyronuronate (9): 3.75 g (7.73 mmol) sugar 7 and 189 mg (1.55 mmol) DMAP in 50 ml dry CH₂Cl₂ under N₂ were subjected to 2.51 g (15.5 mmol) 1,1′-carbonyl-diimidazole. When the reaction was complete by TLC (silica, 5% MeOH/CH₂Cl₂) (2.25 h), the solution was washed with water, 5% NaH₂PO₄, sat. NaHCO₃, and brine. The organic layer was dried (MgSO₄) and concentrated in vacuo to yield 4.19 g of the imidazolyl intermediate 8.8 was dissolved in 65 ml anhydrous CH₂Cl₂ under N₂ and cooled to 0° C. 0.90 ml (7.95 mmol) MeOTf was added over 5 min. After 30 min, the reaction was diluted with 30 ml Et₂O and cooled to −20° C. to allow all methylated product to precipitate. The white solid was collected by filtration, washed with Et₂O and dried in vacuo. The activated compound was suspended in 50 ml anhydrous CH₂Cl₂ under N₂ and 2.22 g (10.85 mmol) 2-bromoethylamine hydrobromide were added. The slurry was brought to 0° C. and 1.51 ml (10.85 mmol) TEA added in one portion. The reaction stirred for 2 h and was then washed with water and brine. The organic layer was dried (MgSO₄), concentrated in vacuo and purified by chromatography (silica, 0-55% EtOAc in hexanes) to give 2.99 g (65%) 9 as a white solid. ¹H NMR (400 MHz, DMSO-d₆) δ 2.00 (s, 3H, OAc), 2.02 (s, 6H, OAc), 3.39 (t, 2H, J=6 Hz), 3.47 (t, 2H, J=6 Hz), 3.64 (s, 3H, COOCH₃), 4.73 (d, 1H, H-5, J=10 Hz), 5.06-5.14 (m, 4H, benzylic CH₂, H-2, H-4), 5.46 (m, 1H, H-3), 5.74 (d, 1H, H-1 J=8 Hz), 7.43 (d, 1H, ArH, J=9 Hz), 7.67 (m, 2H, ArH, NH), 7.89 (s, 1H, ArH); C NMR (101 MHz, DMSO-d₆) δ 20.22, 20.26, 20.31, 32.44, 42.34, 52.63, 63.81, 68.65, 69.83, 70.66, 71.01, 97.79, 117.72, 123.86, 132.77, 133.51, 139.99, 147.58, 155.70, 166.78, 168.62, 169.21, 169.38; ESI-MS m/z (M+Na)⁺657.0, 659.0 (Br isotope pattern); Anal. Calcd for C₂₃H₂₇BrN₂O₁₄: C 43.48, H 4.28, N 4.41; Found C, 43.74; H, 4.20; N, 4.34.

Methyl 1-(4-(2-bromo-ethylcarbamoyloxymethyl)-2-nitrophenyl)-β-D-glucopyronuronate (10): 2.84 g (4.47 mmol) 9 were suspended in 90 ml dry MeOH under N₂ at 0° C. 650 μL 30% w/v NaOMe in MeOH were added and the solution stirred for 100 min. The reaction was quenched with 197 μL acetic acid. Removal of solvent was followed by purification on silica (10% MeOH/CH₂Cl₂). The resulting solid was dissolved in acetone and filtered through a 0.2 μm PTFE filter to remove any silica. The solution was concentrated in vacuo and triturated (Et₂O) to give 1.80 g (79%) 10. Data for 10 ¹H NMR (500 MHz, DMSO-d₆) δ 3.26-3.42 (m, 5H), 3.47 (t, 2H, J=6 Hz), 3.65 (s, 3H, COOCH₃), 4.13 (d, 1H, H-5, J=10 Hz), 5.04 (s, 2H, benzylic CH2), 5.31 (m, 2H, H-1, OH), 5.49 (d, 1H, OH J=6 Hz), 5.54 (d, 1H, OH J=5 Hz), 7.44 (d, 1H, ArH, J=9 Hz), 7.63 (m, 2H, ArH, NH), 7.86 (s, 1H, ArH); ¹³C NMR (126 MHz, DMSO-d₆) δ 32.44, 42.35, 52.02, 63.94, 71.19, 72.71, 75.17, 75.61, 99.76, 116.86, 124.09, 131.27, 133.54, 139.86, 148.58, 155.88, 169.06; ESI-MS m/z (M+Na)⁺530.9, 532.9 (Br isotope pattern); Anal. Calcd for C₁₇H₂₁BrN₂O₁₁: C 40.09; H 4.16; N 5.50; Found C 40.32; H 4.46; N 5.39. The major byproduct of this reaction was α,β-unsaturated compound 11, resulting from base-catalyzed β-acetate elimination. Data for 11 ¹H NMR (500 MHz, CD₃OD) δ 3.23 (t, 2H, J=5 Hz), 3.45 (t, 2H, J=5 Hz), 3.77 (s, 3H, COOCH 3), 4.09 (m, 2H), 5.09 (s, 2H, benzylic CH₂), 5.90 (br. s, 1H, H-4), 6.28 (d, 1H, H-1 J=2.5 Hz), 7.57 (d, 1H, ArH, J=9 Hz), 7.66 (d, 1H, ArH, J=9 Hz), 7.92 (s, 1H, ArH); ¹³C NMR (126 MHz, CD₃OD) δ 33.67, 44.41, 53.12, 65.83, 66.77, 70.58, 100.08, 114.14, 120.19, 126.04, 134.37, 135.11, 141.05, 142.02, 150.24, 158.73, 163.96.

Methyl 1-(4-(2-(1-(1,4,7,10-tetrazaacyclododecyl))-ethylcarbamoyloxymethyl)-2-nitrophenyl)-β-D-glucopyronuronate (12): Cyclen (541 mg, 3.14 mmol) and 10 (640 mg, 1.26 mmol) were combined in 19 ml DMSO and the reaction allowed to stir overnight. TLC analysis at this time (10% MeOH/CH₂Cl₂) indicated no unreacted sugar. The solvent was removed in vacuo yielding a viscous yellow oil. The oil was dissolved in 7 ml MeOH and a pale yellow solid precipitated upon addition of 50 ml Et₂O. Upon storage at −20° C. for 1 h, the hygroscopic solid was collected on a glass frit, washed with Et₂O(3×3 ml) and dried under vacuum yielding 908 mg of solid. TLC (silica; 1:9:90 sat KNO₃(aq.):water:MeCN; Pt stain visualization) and ESI-MS showed very low-intensity di- and tri-substituted side product peaks. The precipitation procedure removed excess free base cyclen, however MS and H NMR showed that the desired product was contaminated with cyclen hydrobromide salt. This mixture was used in the subsequent reaction without further purification.

Methyl 1-(4-(2-(1-(4,7,10-tris-ethylcarboxymethyl-(1,4,7,10-tetrazaacyclododecyl)))-ethylcarbamoyloxymethyl)-2-nitrophenyl)-β-D-glucopyronuronate (13): 887 mg of the mixture containing 12 and 1.23 g K₂ CO were suspended in 30 ml acetone. 820 μl α-bromo-ethylacetate were added and the solution was allowed to stir at room temperature overnight. An additional 164 μl α-bromo-ethylacetate and 210 mg K₂CO₃ were added after 24 h. At 48 h, the reaction mixture was filtered to remove solids and purified by flash chromatography (silica, 0-13.3% MeOH in CH₂Cl₂). The resulting solid was dissolved in acetone and filtered through a 0.2 μm PTFE filter to remove excess silica. This yielded 510 mg of 13. Elemental bromine analysis indicated the presence of a mixture of free base and hydrobromide salt. ¹H NMR (500 MHz, CD₃OD) δ 1.25 (m, 9H, COOCH₂CH₃), 2.0-3.4 (br, 24H, cyclen H's, 2H-sugar, acetate CH₂), 3.52 (m, 4H), 3.64 (m, 1H), 3.76 (s, 3H, COOCH₃), 4.10 (d, 1H, J=10 Hz), 4.12-4.24 (m, 6H, COOCH₂CH₃), 5.07 (s, 2H, benzylic CH₂), 5.21 (d, 1H, H-1, J=7 Hz), 7.39 (d, ArH, J=8 Hz), 7.60 (d, 1H, ArH, J=8 Hz), 7.83 (s, 1H, ArH); ¹³C NMR (126 MHz, CD₃OD) δ 14.64, 14.67, 38.73, 53.10, 55.99, 56.44, 56.88 (br), 62.61, 62.83, 66.01, 72.79, 74.46, 76.90, 77.25, 102.30, 118.81, 125.33, 133.50, 134.31, 142.19, 150.54, 159.14, 170.76, 175.57, 175.66 (br). ESI-MS m/z (M+H)⁺859.2 (40%), (M+Na)⁺881.2 (100%); Anal. Calcd for C₃₇H₅₈N₆O₁₇. acetone.2.5H₂O.0.75HBr: C 46.98, H 6.87, N 8.22, Br 5.86; Found C 47.05; H 6.55; N 8.23, Br 6.07.

Gadolinium(III) 1-(4-(2-(1-(4,7,10-tris-carboxymethyl-(1,4,7,10-tetrazaacyclododecyl)))-ethylcarbamoyloxymethyl)-2-nitrophenyl)-β-D-glucopyronuronate (1): 455 mg 13 in 10 ml water were cooled to 0° C. 2.12 ml 1 N NaOH were added over one minute and the solution was allowed to stir for 75 min. The pH was brought to 6.5 with 0.1 N HCl and 216 mg GdCl₃ (dissolved in 5 ml water and brought to pH=6.5 with NaOH) were added dropwise. The pH was kept above 5.5 during metal addition with 1 N NaOH. The solution was allowed to warm to room temperature while stirring and the pH adjusted periodically to keep it between 6-6.5. After 3 days at room temperature, the pH showed no change and the reaction was considered complete. The pH was brought to 8.2 and the solution centrifuged to remove excess gadolinium as Gd(OH)₃. Trace solids were removed by filtration through a 0.2 μm nylon filter and the solution lyophilized. The solid was brought up in 3 ml water and purified on preparative HPLC using the following method: 0-10% B over 10 min, hold for 15 min at 10% B, then wash to 98% B before returning to 0% B. Two runs using this method were sufficient to give material that was pure by microanalysis. Yield: 185 mg 1 (17.7% from 10). The compound was stored at −20° C. Analysis of this material by analytic LC-MS (using the same method as in the preparative runs) gave a single peak in the PDA at 12.9 min with an m/z=914.4 (M−H⁺ ESI-MS) of appropriate isotope pattern. Calcd for C₃₀H₄₀N₆O₁₇Gd.2.5H₂O (87% Na⁺ salt): C 37.85; H 4.46; N 8.83, Gd 16.52, Na 2.10; Found C 37.69; H 4.28; N 9.12, Gd 16.88, Na 2.10.

Europium(III) 1-(4-(2-(1-(4,7,10-tris-carboxymethyl-(1,4,7,10-tetrazaacyclododecyl)))-ethylcarbamoyloxymethyl)-2-nitrophenyl)-β-D-glucopyronuronate (4): This compound was synthesized and purified in the same manner as 1 using 168 mg 13 and substituting EuCl₃ for GdCl₃. Yield: 70 mg 4 (18.1% from 10). The compound was stored at −20° C. Analysis of this material by analytic LC-MS (using the same method as in the preparative runs) gave a single peak in the PDA at 12.9 min with an m/z=907.2 (M−H⁺ ESI-MS) of appropriate isotope pattern.

Methyl 1-(4-(2-hydroxy-ethylcarbamoyloxymethyl)-2-nitrophenyl)-2,3,4-tri-O-acetyl-β-D-glucopyronuronate (15): 1.05 ml (9.25 mmol) MeOTf were added over 5 min to a solution of 4.87 g (8.41 mmol) 8 in 60 ml anhydrous CH₂Cl₂ under N₂ at 0° C. After 30 min, the reaction was diluted with 30 ml Et₂O and cooled to −20° C. to allow all methylated product to precipitate. The white solid was collected by filtration, washed with Et₂O and dried in vacuo. The activated compound was suspended in 60 ml anhydrous CH₂Cl₂ under N₂ and brought to 0° C. 761 μl (12.6 mmol) 2-hydroxyethylamine were added and the solution was allowed to warm to room temperature over 2 h and was then washed with water, 5% NaH₂PO₄, sat. bicarbonate and brine. The organic layer was dried (MgSO₄), concentrated in vacuo and purified by chromatography (silica, 0-5% MeOH in CH₂Cl₂) to give 3.68 g (77%) 15 as a white solid. ¹H NMR (500 MHz, DMSO-d₆) δ 1.99 (s, 3H, OAc), 2.02 (s, 6H, OAc), 3.05 (q, 2H, J=6 Hz), 3.33 (s, 1H, OH), 3.38 (q, 2H, J=6 Hz), 3.64 (s, 3H, COOCH₃), 4.64 (t, 0.5H, NH, J=6 Hz), 4.74 (d, 1H, H-5, J=10 Hz), 5.02 (s, 2H, benzylic CH₂), 5.10 (m, 2H), 5.46 (t, 1H, J=10 Hz), 5.74 (d, 1H, H-1, J=8 Hz), 7.27 (t, 0.5H, NH, J=6 Hz), 7.43 (d, 1H, ArH, J=9 Hz), 7.67 (d, 1H, ArH, J=9 Hz), 7.88 (s, 1H, ArH); ¹³C NMR (126 MHz, DMSO-d₆) δ 20.20, 20.23, 20.28, 43.09, 52.64, 59.86, 63.59, 68.69, 69.87, 70.71, 71.05, 97.89, 117.80, 123.89, 133.12, 133.55, 140.11, 147.63, 155.93, 166.90, 168.74, 169.33, 169.51;IR(KBr plate) v 3394, 2962, 1756, 1708, 1535, 1366, 1235, 1073, 1039 cm⁻¹; ESI-MS m/z (M+Na)⁺ 595.3; Anal. Calcd for C₂₃H₂₈N₂O₁₅: C 48.25, H 4.93, N 4.89; Found C 47.96; H 5.14; N 4.91.

Methyl 1-(4-(2-hydroxy-ethylcarbamoyloxymethyl)-2-nitrophenyl)-β-D-glucopyronuronate (16): 3.49 g (6.10 mmol) 15 in 100 ml anhydrous MeOH were cooled to 0° C. under N₂. 776 μl (4.27 mmol) 30% NaOMe in MeOH were added and the solution allowed to stir for 1 h. 210 μl acetic acid were added and the volatiles removed in vacuo. The resulting solid was purified by chromatography (silica, 10-15% MeOH in CH₂Cl₂) and excess silica removed by filtration of an acetone solution through a 0.2 μm PTFE filter. Trituration of the solid with Et₂O yielded 2.15 g (79%) 16 as a white solid. ¹H NMR (500 MHz, DMSO-d₆) δ 3.05 (q, 2H, J=6 Hz), 3.25-3.44 (m, 6H), 3.66 (s, 3H, COOCH₃), 4.12 (d, 1H, H-5, J=10 Hz), 4.63 (t, 0.5H, NH, J=5.5 Hz), 5.00 (s, 2H, benzylic CH₂), 5.31 (d, 2H, H-1, OH), 5.49 (d, 1H, OH, J=5.5 Hz), 5.54 (d, 1H, OH, J=4.5 Hz), 7.25 (t, 0.5H, NH, J=6 Hz), 7.44 (d, 1H, ArH, J=9 Hz), 7.63 (d, 1H, ArH, J=9 Hz), 7.85 (s, 1H, ArH); ¹³C NMR (126 MHz, DMSO-d₆) δ 43.10, 52.03, 59.88, 63.70, 71.20, 72.72, 75.17, 75.61, 99.80, 116.86, 124.05, 133.51, 133.51, 139.87, 148.53, 155.99, 169.07; IR (KBr plate) v 3352, 2954, 1737, 1705, 1533, 1354, 1252, 1083, 1060, 1019 cm⁻¹; ESI-MS m/z (M+Na)⁺ 469.2; Anal. Calcd for C₁₇H₂₂N₂O₁₂: C 45.74; H 4.97; N 6.28; Found C 47.92; H 5.06; N 5.98.

Methyl 1-(4-(2-methanesulfonyloxy-ethylcarbamoyloxymethyl)-2-nitrophenyl)-β-D-glucopyronuronate (14): 950 μl (6.81 mmol) NEt₃, 100 mg (0.85 mmol) DMAP and 1.90 g (4.26 mmol) 16 were dissolved in 50 ml anhydrous pyridine and cooled to 0° C. 529 μl (6.81 mmol) methanesulfonyl chloride were added and the reaction checked by TLC (10% MeOH/CH₂Cl₂). After 1 h an additional 0.5 eq (2.13 mmol) of NEt₃ and MsCl were added and the solution was allowed to stir for 1 h more. The volatiles were then removed in vacuo and the resulting oil purified by chromatography (silica, 10% MeOH/CH₂Cl₂). The solid was dissolved in acetone, filtered as in 16, concentrated and triturated with hexanes to give 1.60 g (72%) 14. The compound decomposes upon prolonged storage at ambient temperature even desiccated under N₂. ¹H NMR (500 MHz, CD₃OD) δ 3.03 (s, 3H), 3.44 (m, 2H), 3.48-3.66 (m, 3H, H2-4), 3.76 (s, 3H, COOCH₃), 4.09 (d, 1H, H-5, J=10 Hz), 4.25 (m, 2H), 5.09 (s, 2H, benzylic CH₂), 5.19 (d, 1H, H-1), 7.37 (d, 1H, ArH, J=8 Hz), 7.59 (d, 1H, ArH, J=8 Hz), 7.83 (s, 1H, ArH); ¹³C NMR (126 MHz, CD₃OD) δ 637.32, 41.37, 53.11, 66.06, 69.95, 72.77, 74.48, 76.89, 77.25, 102.44, 118.94, 125.64, 133.44, 134.52, 142.24, 150.61, 158.62, 170.81; Anal. Calcd for C₁₈H₂₄N₂O₁₄S: C 41.22; H 4.61; N 5.34; Found C 41.52; H 4.55; N 5.24.

1-(2-^{t}Boc-aminoethyl)-(1,4,7,10-tetrazaacyclododecane) (17): 1.0 g (4.46 mmol) 2-^{t}Boc-aminoethylbromide were added to a stirring solution of 1.92 g (11.1 mmol) cyclen in 60 ml dry toluene. The solution was refluxed overnight under N₂ and extracted with 3×100 ml water. The aqueous layer was extracted with 3×75 ml CH₂Cl₂ and the combined CH₂Cl₂ extracts were dried over MgSO₄. Removal of solvent gave a white solid that was washed with cold Et₂O and dried in vacuo. This yielded 890 mg (63%) 15. ¹H NMR (500 MHz, CDCl₃) δ 1.44 (s, 9H), 2.59 (br s, 10H), 2.63 (br s, 4H), 2.82 (br s, 4H), 3.22 (br s, 2H); ¹³C NMR (126 MHz, CDCl₃) δ 28.58, 38.65, 46.21, 47.28, 47.92, 52.20, 54.28, 78.96, 156.14; ESI-MS m/z (M+H)⁺ 316.3; Anal. Calcd for C₁₅H₃₃N₅O₂: C 57.11; H 10.54; N 22.20; Found C 57.43; H 10.47; N 22.51.

1-(2-^{t}Boc-aminoethyl)-4,7,10-(tris-^{t}butylcarboxymethyl)-(1,4,7,10-tetrazaacyclododecane) (18): To a solution of 950 mg 17 (3.01 mmol) and 3.29 g (31.0 mmol) Na₂CO₃ in dry MeCN under N₂, was added 2.4 ml (15.1 mmol) α-bromo-^{t}butylacetate. The suspension was refluxed for 24 h, filtered, washed with 3×250 ml hexanes and concentrated in vacuo to give a yellow oil. The resulting oil was purified by chromatography (silica, 0-10% MeOH in CH₂C₂) to give 1.70 g (76%) of 18 as a white solid. Spectral and analytic data indicate a mixture of free base and HBr salt. The ¹H NMR was very broad between 2-3.8 ppm and therefore unasssignable; ¹³C NMR (126 MHz, CDCl₃) δ (major product) 27.73, 27.92, 28.03, 28.32, 37.69, 48.01, 49.88, 50.09, 52.50, 53.01, 53.82, 55.60, 56.40(br), 56.91, 79.17, 81.70, 82.74, 156.40, 169.99, 172.51, (minor peaks): 79.30, 81.80, 82.36, 170.33, 173.28(br); ESI-MS m/z (M+H)⁺ 658.4 (60%), (M+Na)⁺ 680.3 (100%); Anal. Calcd for C₃₃H₆₃N₅O₈.0.9HBr.H₂O: C 52.94, H 8.87, N 9.35, Br 9.60; Found C 52.81; H 8.99; N 9.02, Br 9.78.

1-(2-aminoethyl)-4,7,10-(tris-carboxymethyl)-(1,4,7,10-tetrazaacyclododecane) TFA salt (19): Deprotection of 192 mg 18 was achieved by stirring at room temperature in 4.75 ml trifluoroacetic acid with 125 μl each triisopropylsilane and water. After 17 h the volatiles were removed in vacuo and 40 ml Et₂O were added to precipitate the ligand. The suspension was centrifuged and the white pellet washed with 3×50 ml Et₂O. The resulting solid was dried under vacuum and yielded 135 mg of the TFA salt, 19. ¹H NMR showed no remaining ^{t}butyl resonances while ¹⁹F NMR showed a signal for TFA. ESI-MS m/z (M+H)⁺ 390.2.

Gadolinium(III)1-(2-aminoethyl)-4,7,10-(tris-carboxymethyl)-(1,4,7,10-tetrazaacyclododecane) (2): 128 mg (0.61 mmol) Gd(OH)₃.H₂O and 239 mg 19 were combined in 10 ml water and the suspension refluxed for 48 h. The solution was brought to pH=10 with conc. NH₄OH and centrifuged to remove excess Gd(OH)₃. The pellet was washed and the combined washings and supernatant were lyophilized. The resulting solid was dissolved in water and purified by successive runs on preparative HPLC using the following method: 0-20% B over 10 min, hold at 20% B for 15 min, then wash to 98% B before returning to 0% B. Due to lack of chromophores, the compound displays little UV absorption, fluorescence however can be detected by exciting at 271 nm and monitoring the emission at 314 nm. Due to peak tailing, fractions were analyzed by analytic LC-MS and those containing 2 were pooled and lyophilized. 101 mg 2 were obtained analytically pure by this approach (40% from 18). ESI-MS m/z (M+Na)⁺ 567.0 with Gd isotope pattern. Anal. Calcd for C₁₆H₂₈N₅O₆Gd.H₂O: C 34.21; H 5.38; N 12.47; Found C 34.16; H 5.31; N 12.08.

Europium(III)1-(2-aminoethyl)-4,7,10-(tris-carboxymethyl)-(1,4,7,10-tetrazaacyclododecane) (3): 128 mg 19 and 132 mg (0.36 mmol) EuCl₃.6H₂O were combined in water and the pH adjusted to 6 with 1 N NaOH. The reaction was stirred for 3 days at room temperature, filtered and lyophilized. The freeze-dried solid was purified in the same manner (and exhibited similar peak tailing) as 2 except fluorescence detection used λₑₓ=395 nm and λₑₘ=615 nm. 45 mg 3 were obtained analytically pure in this fashion (33% from 18). ESI-MS m/z (M+Na)⁺ 559.8, 561.7, Eu isotope pattern. Anal. Calcd for C₁₆H₂₈N₅O₆Eu.0.5H₂O: C 35.11; H 5.34; N 12.79, Eu 27.76; Found C 35.03, H 5.41, N 12.54, Eu 27.89.

Relaxivity

A 4 mM stock solution of either 1 or 2 in the appropriate buffer was diluted to give 500 μL each of seven approximate concentrations for each run: 0, 0.05, 0.15, 0.3, 0.5, 1.0, and 2.0 mM. The T₁ of each concentration was determined using an inversion recovery pulse sequence with appropriate recycle delays on a Bruker mq60 Minispec. This instrument has a proton Larmor frequency of 60 MHz and operates at 37° C. The resulting curves were fit to a monoexponential function to obtain T₁0.10 μL of each sample was digested in concentrated nitric acid, diluted with water and analyzed for exact Gd(III) concentration using ICP-MS. The reciprocal of the longitudinal relaxation time was plotted against the concentration obtained from ICP-MS and fit to a straight line. All lines fit with R²>0.998. This was performed for each buffer in duplicate. The buffers were all made to have the appropriate pH at 37° C. and remade if the pH had drifted more than 0.05 pH units upon storage. Anion mimic and carbonate containing buffers were made fresh daily.

Enzyme Kinetics

General: Bovine liver β-glucuronidase (Type B-1, Sigma G 0251) stability in several buffers was assayed by incubating the enzyme in the desired buffer and sampling its activity at various time intervals using the Sigma quality control assay for β-glucuronidase from bovine liver. It was determined that MOPS and anion mimic (See, e.g., Parker, D. In Crown Compounds: Towards Future Applications; Cooper, S. R., Ed.; VCH: New York, 1992, pp 51-67) engendered the enzyme with poor stability, while 100 mM phosphate with 0.01% (w/v) bovine serum albumin (BSA), pH=7.4 at 37° C. gave suitable stability (>2 h at 37° C. without loss of activity) provided the enzyme concentration was greater than 0.5 mg/ml. The enzyme was stable for at least 24 h at 37° C. at its native pH of 5.0 in 100 mM acetate buffer. All kinetics measurements were made with 0.2 mM substrate, 1, in 510 μL buffer with either 1.0 mg/ml enzyme (those buffers at pH=7.4) or 0.1 mg/ml enzyme (pH=5.0 acetate buffer). For all buffers except serum (due to its opacity), the kinetics were determined simultaneously by both UV-visible and magnetic resonance. The runs were all continuous assays that are known to give the best data (See, e.g., Marangoni, A. G. Enzyme kinetics: a modern approach; Wiley-Interscience: Hoboken, N.J., 2003; Copeland, R. A. Enzymes: a practical introduction to structure, mechanism, and data analysis; VCH Publishers: New York, N.Y., 1996). Each run was performed in triplicate and two controls, one without enzyme and one without substrate, were also measured. In all instances with the exception of the magnetic resonance substrate control in acetate buffer, the controls showed very little change over the 1 h duration of each experiment. The substrate in acetate buffer showed a slight (2%) decrease in T₁ over the course of an hour. This trend is in the opposite direction of the kinetics runs that show an increase in T₁.

LC-MS. After 2 h, the reaction mixture was analyzed by LC-MS and showed the presence of 4-hydroxy-3-nitrobenzyl alcohol at 4.0 min, substrate 1 at 7.5 min, and 2 at 11.8 min. The alcohol and 1 are readily distinguished by their absorption spectra (PDA), their appropriate negative mode ESI-MS patterns and through spiking with authentic compound. 2 was more difficult to detect but could be observed using fluorescence. The HPLC method was as follows: 0-10% B over 10 min, hold at 10% B for 15 min, with fluorescence using λₑₓ=271 nm, λₑₘ=314 nm.

UV-visible: The enzymatic hydrolysis of the pyranose from 1 and subsequent linker decomposition generates 4-hydroxy-3-nitrobenzyl alcohol. This compound has a maximum absorbance of 422 nm at pH=7.4. The molar absorptivity, E, was determined in triplicate from 0-0.2 mM, on an HP 8452A diode array spectrometer thermostated to 37° C. For 100 mM phosphate, 0.01% (w/v) BSA, pH=7.4 at 37° C. it is 2840±40 M⁻¹ cm⁻¹. For 100 mM phosphate, 24 mM NaHCO₃, 0.01% (w/v) BSA, pH=7.4 at 37° C. it is 3010±50 M⁻¹ cm⁻¹. At pH=7.4, the substrate, 1, does not absorb at this wavelength. At pH=5.0 in acetate buffer, the maximum is at 354 nm. This overlaps with substrate, 1, absorption. The kinetics were sampled on the HP 8452A at 37° C. every 20 seconds for an hour. The initial rates for pH=7.4 buffers were determined through a linear fit of the first 10% change in absorbance. For the acetate buffer, only a half-life of conversion is reported. Analysis of the reaction mixture by LC-MS using the method detailed in purification of 1 after 1 h confirmed the presence of 4-hydroxy-3-nitrobenzyl alcohol and compound 2. 4-hydroxy-3-nitrobenzyl alcohol was also present in the acetate runs as determined by LC-MS using 100 mM acetate pH=5 as solvent A.

Magnetic Resonance: T₁ was determined at intervals of 2 minutes for the first 30 min and 4 minutes for the next 30 min using a saturation recovery (90-T-90) pulse sequence using the Bruker mq60 operating as detailed in the relaxivity section. This sequence is less accurate than the inversion recovery method, but gives faster results. The runs were performed in triplicate. The substrate-only control was also examined in this manner. The enzyme-only controls showed T₁'s that were identical to neat buffer.

Determination of q: Europium(III) compound 4 was dissolved in H₂O and D₂O. The emission was monitored at 614 nm with excitation at 394 nm on a Hitachi F4500 fluorometer operating in Phosphorescence Lifetime (short) mode. The shortest lifetime measurable with this instrument is about 0.3 ms. Fifteen scans were averaged and fit to a monoexponential decay to give phosphorescent lifetimes. T(D₂O)=1.274 ms; T (H₂O)=0.527 ms. Using the equation of Supkowski and Horrocks (See, e.g., Supkowski and Horrocks, Inorg. Chim. Acta 2002, 340, 44-48), q=0.89 while the equation from Beeby et al. ⁴⁰generates q=1.04.

Viscosity: Determinations were made using a Gilmont Instruments model GV-2100 falling ball viscometer held at 37° C. using a recirculating water bath. Time measurements were performed in quintuplicate, averaged and plugged into the equation supplied by the manufacturer. This equation also requires the density of the solution under scrutiny. The density was determined by weighing 1000 μL of solution at 37° C. The error in this measurement performed in triplicate was ten-fold less than the time determination and was not propagated. The values reported represent the mean of the five time determinations with an error of one standard deviation. The presence of solids in the human serum precluded determination of its viscosity.

### Example 2

### Synthesis

Scheme 1. The application of β-glucuronide prodrugs in prodrug monotherapy (PMT) has yielded mixed results (See, e.g., Bosslet et al., Cancer Res. 1998, 58, 1195-1201; Guerquin-Kern et al., NMR Biomed. 2000, 13, 306-310). In PMT, β-glucuronic acid is liberated from the relatively non-toxic prodrug via endogenous extracellular β-glucuronidase yielding the more potent chemotherapeutic. The drawbacks to this approach are that high enzyme levels are found only near necrotic tumor masses that have low perfusion and hence receive less prodrug and that enzyme concentration is variable between individuals (See, e.g., Rooseboom et al., Pharmacol. Rev. 2004, 56, 53-102; Brusselbach, S. Methods in Molecular Medicine 2004, 90, 303-330). Further complicating matters is the short half-life of glucuronide conjugated prodrugs, necessitating fast enzymatic conversion of the prodrug to its active form (See, e.g., Guerquin-Kem et al., NMR Biomed. 2000, 13, 306-310). Antibody directed enzyme prodrug therapy (ADEPT; a two step approach), introduces exogenous enzyme via an antibody targeting moiety and in principle, should overcome the problems associated with PMT. ADEPT progress, initially curtailed by host immune response to the antibody-enzyme conjugate, has shown some promise with the advent of antibodies engineered via phage display (See, e.g., Pedley et al., Methods in Molecular Medicine 2004, 90, 491-514). Another potential avenue involves gene-directed enzyme prodrug therapy (GDEPT). Here, the diseased cells are transfected with DNA coding for the enzyme that is then produced by the cell and effects the prodrug cleavage. The cell surface display of β-glucuronidase has recently been reported as a candidate for GDEPT (See, e.g., Heine et al., Gene Therapy 2001, 8, 1005-1010).

Thus, In order to optimize the enzyme cleavage kinetics of 1, the present invention provides the incorporation of a nitrophenyl self-immolative linker. The kinetics of previous galactosidase sensitive agents (EGad and EGadMe) (See, e.g., Moats et al., Chem., Int. Ed. Engl. 1997, 36, 726-728; Louie et al., Nat. Biotechnol. 2000, 18, 321-325) were impedingly slow, prompting discovery of the compositions and methods provided by the present invention. Thus, in some embodiments, the present invention provides an MR contrast agent that is modulated by changing q, the number of inner-sphere, Gd(III) coordinated water molecules. The use of a linker longer than the hydroxyethyl structure used in EGad (See, e.g., FIG. 3B) may preclude efficient water blockage by the sugar. However, the seven-coordinate DO3A analogs have reduced relaxivities due to coordination of endogenous bidentate anions such as carbonate (See, e.g., Bruce et al., J. Am. Chem. Soc. 2000, 122, 9674-9684; Dickins et al., J. Am. Chem. Soc. 2002, 124, 12697-12705; Messeri et al., Chem. Comm. 2001, 2742-2743; Supkowski et al., Inorg. Chem. 1999, 38, 5616-5619). Thus, the present invention provides the seven coordinate chelate structure of 1 that allows bidentate anion binding to occur. In some embodiments, upon enzymatic cleavage, the aminoethyl arm binds the metal center expelling the anion and generating an octadentate center with q=1, thus creating compound 2 (See, e.g., FIG. 3A). Octadentate complexes such as 2 bind anions with a much lower affinity (See, e.g., Supkowski et al., Inorg. Chem. 1999, 38, 5616-5619; Burai et al.,. Mag. Reson. Med. 1997, 38, 146-150) (3 orders of magnitude for carbonate). Thus, it is contemplated that, in some embodiments, water access should approach that of a q=1 complex. Therefore, in some embodiments, 2 has a higher relaxivity than the nominally q=0 1 in the presence of endogenous anions and the agent goes from low relaxivity (dark) to high relaxivity (bright) in the presence of β-glucuronidase.

The original investigations involving the β-galactosidase activated agents, EGad, 4, and EGadMe, 5, (See, e.g., FIG. 3B) used an “aqueous” synthetic route to obtain the desired complexes (See, e.g., Moats et al., Chem., Int. Ed. Engl. 1997, 36, 726-728; Louie et al., Nat. Biotechnol. 2000, 18, 321-325). In this scheme, the sugar/cyclen conjugate was deprotected in aqueous methanol and the acetate arms were added in alkaline water. The complex was isolated from this mixture using anion exchange. The large quantities of ammonium acetate used for ion exchange proved difficult to completely remove and hindered the subsequent metallation reaction. Furthermore, the one-pot approach did not permit comprehensive characterization of intermediates. For these reasons, an “organic” synthetic route to the more complex compound 1 was employed (See, e.g., Schemes 2-6, below). This procedure permitted facile characterization with increased reproducibility.

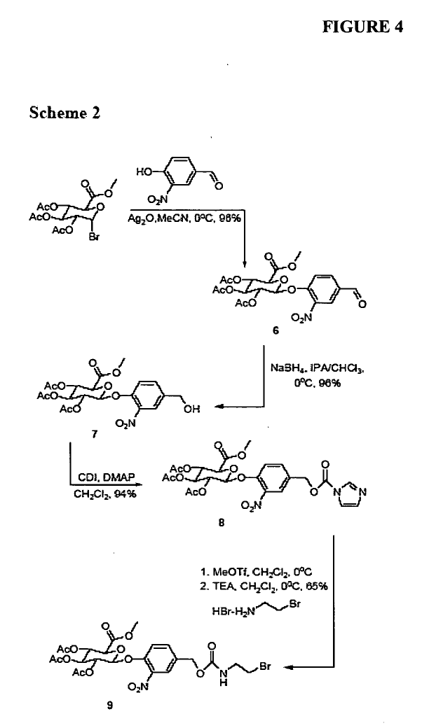

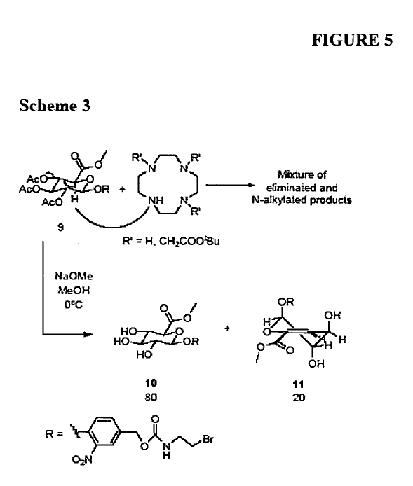

Scheme 2. The synthesis of 1 began with methyl 1-bromo-2,3,4-tri-O-acetyl-α-D-glucopyronuronate (See, e.g., Bollenback et al., A. J. Am. Chem. Soc. 1955, 77, 3310-3315, and FIG. 4, Scheme 2). Coupling of 4-hydroxy-3-nitrobenzaldehyde to the pyranose via a Koenigs-Knorr reaction followed by sodium borohydride reduction produced 7 in 92% yield without recourse to chromatography. Initial attempts at formation of the carbamate using p-nitrophenyl chloroformate (See, e.g., Florent et al., J. Med. Chem. 1998, 41, 3572-3581; Leu et al., J. Med. Chem. 1999, 42, 3623-3628) and triphosgene (See, e.g., Eckert et al., Chem. 1987, 99, 922-923; Majer and Randad, J. Org. Chem. 1994, 59, 1937-1938) gave intermediates that showed insufficient reactivity towards 2-bromoethylamine and 2-hydroxyethylamine. The reaction occurred smoothly however when carbonyl-diimidazole (CDI) was used as the carbonyl equivalent. Methylation of the mono-imidazolyl intermediate gave an increased yield through precipitation of the cationic intermediate. Alkylation reactions involving 9 with either cyclen or DO3A(tris-^{t}butyl ester) (See, e.g., Dadabhoy et al., J. Chem. Soc., Perkin Trans. 2 2002, 348-357) generated large amounts of α-β-unsaturated byproducts due to an acid-base reaction between cyclen and the acidic proton alpha to the methyl ester (See, Scheme 3, FIG. 5). It is known that this type of elimination can happen with acetyl protected glucuronic acids (See, e.g., Schmidt and Neukom, Tetrahedron Lett. 1969, 2011-2012; Stachulski and Jenkins, Nat. Prod. Rep. 1998, 15, 173-186) but it is rarely mentioned in the literature. Synthesis of methyl 1-ethoxy-2,3,4-tri-O-acetyl-β-D-glucopyronuronate and subsequent monitoring of the reaction with cyclen by ¹H NMR confirmed the elimination. The formation of the α,β-unsaturated byproducts was circumvented by selective removal, in good yield, of the acetyl groups from 9 using sodium methoxide, with 11 as the byproduct (See, e.g., FIG. 5, Scheme 3). Introduction of the macrocycle was then attempted through two approaches.

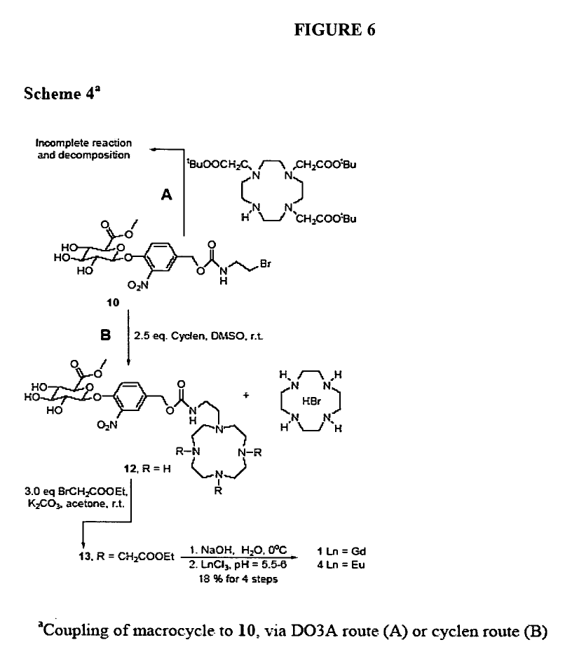

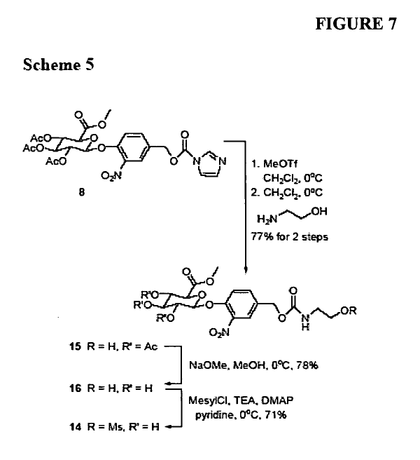

In the first approach, DO3A(tris-^{t}butyl ester) was reacted with 10 (See, e.g., FIG. 6, Scheme 4, A). After several days at room temperature, the reaction was not complete and byproducts had begun to develop. Attempts involving heating of the reaction induced sugar decomposition. The cyclen route in which the sugar containing arm is added prior to the acetate arms (See, FIG. 6, Scheme 4, B), was successful, presumably because the macrocycle free-base is more nucleophilic towards the unactivated alkyl bromide electrophile than the DO3A compound. Increasing the electrophilicity of 9 (See, e.g., FIG. 4, Scheme 2) by making the mesylated compound, 14 from 8 (See, e.g., FIG. 7, Scheme 5) was not successful due to the propensity of 14 to cyclize via elimination of the mesyl group in the presence of cyclen. Purification of 12 proved difficult, so the compound was alkylated with ethyl bromoacetate. Excess cyclen, as the perethyl DOTA ester, could then removed by chromatography on silica. Final deprotection and metallation were performed in a one-pot procedure. Compound 1 was obtained analytically pure, showing one peak by LC/MS with the appropriate isotope pattern after purification by preparative HPLC. The overall yield for the nine-step procedure was 8.0%. Compound 4 was obtained in 8.2% overall yield by substituting EuCl₃ for GdCl₃.

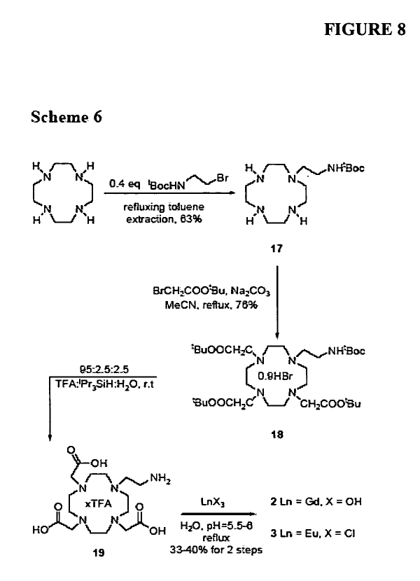

The synthesis of compounds 2 and 3 proved to be more straightforward (See, e.g., FIG. 8, Scheme 6). Utilizing the mono-alklyation of excess cyclen, intermediate 17 was obtained analytically pure following extraction and an ether wash. Subsequent alkylation with three equivalents of ^{t}butyl bromoacetate gave 18 as a 9:1 mixture of hydrobromide salt to free base. Deprotection was achieved through the use of trifluoroacetic acid. Metallation of the free ligand, 19, with Gd(OH)₃ gave crude 2. Purification of 2 via HPLC was difficult for two reasons. The lack of a chromophore on 2 limited detection to fluorescence and the presence of the primary amine ligand gave a peak with a long tail. These difficulties lowered the overall yield to 19% for four steps. The Eu(III) compound 3 was obtained in 16% overall yield by substituting EuCl₃ for Gd(OH)₃. Both compounds were determined to be authentic by elemental analysis.

### Example 3

### Relaxivity

The defining parameter of contrast agent efficacy is relaxivity. In this context, relaxivity, r₁, is a measure of the extent to which the agent, per unit, catalyzes the shortening of the longitudinal relaxation time, T₁, of protons on the hydrogen atoms in bulk water. The presence of other species in solution, be they salts, proteins or small molecules, can have a marked effect on an agent's relaxivity. Relaxivity measurements made in solutions of varying composition not only describe how the agent responds to that composition, but also provide insight into the microscopic processes occurring at or near the Gd(III) center.

Attributing relaxivity effects to the solution composition can be made when the contrast agent under study is of a known purity. Thus, the present invention provides the use of analytically pure contrast agents that allow for facile and accurate determination of agent concentration through the use of Gd(III) ICP-MS. This, in tandem with measurements made in duplicate, reduces the systematic error in the relaxivity measurements.

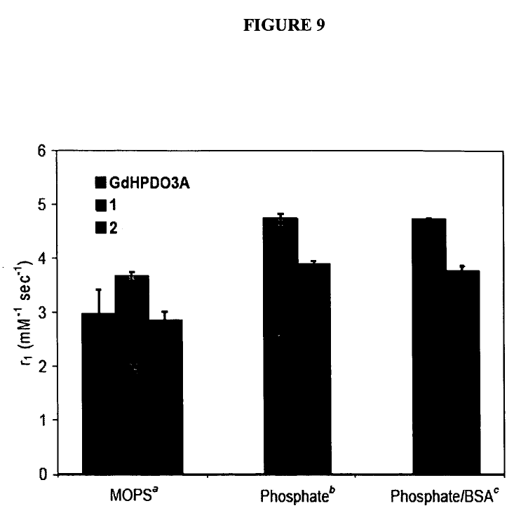

The measurements shown in FIG. 9 reveal trends in relaxivity related to buffer composition. All measurements were made at a proton Larmor frequency of 60 MHz and a temperature of 37° C. To provide a reference, the relaxivity of the known q=1 contrast agent, GdHP-DO3A (Chart 1) was measured in pH=7.4 MOPS buffer and gave an r₁=2.99±0.44 mM⁻¹ sec⁻¹ under these conditions. It has been reported that MOPS does not coordinate to the metal center. Bretonniere and co-workers (See, e.g., Bretonniere et al., Chem. Comm. 2002 1930-1931; Bretonniere, et al., Org. Biomol. Chem. 2004, 2, 1624-1632) perform experiments in MOPS without interference with carbonate binding to Eu(III) trisamide cyclen complexes, while Bruce et al. (See, e.g., Bruce, et al., J. Am. Chem. Soc. 2000, 122, 9674-9684) indicate that experiments with related compounds may be run in the structurally similar HEPES. Initial relaxivity measurements of 1 and 2 made in the same MOPS buffer showed a higher value for 1 and a value comparable to GdHP-DO3A for 2 (FIG. 9, MOPS columns). The magnitude of r₁ for 1 is somewhat low for a q=2 complex however (e.g., since r₁ is directly proportional to q, a q=2 complex should show an r₁ approximately twice that of a q=1 complex such as GdHP-DO3A), indicating intramolecular coordination of the sugar-containing arm to the metal center.

Determination of q via Eu(III) fluorescence (See, e.g., Supkowski and Horrocks, Inorg. Chim. Acta 2002, 340, 44-48; Beeby et al., J. Chem. Soc., Perkin Trans. 2 1999, 493-504) for 4 (the Eu(III) analog of 1) in the absence of buffer salts surprisingly gave a q=1, supporting the intramolecular coordination postulate. Although a mechanism is not needed to practice the present invention, and more than one mechanism is contemplated, it is presumed that this coordination occurs through the carbamate carbonyl oxygen. It is also worth noting that a similar seven-membered intramolecular ring has been invoked to explain low q values for EuDO3A-type complexes containing tethered carboxylates (See, e.g., Messeri et al., Chem. Comm. 2001, 2742-2743; Lowe et al., J. Am. Chem. Soc. 2001, 123, 7601-7609. While the carbamate carbonyl oxygen in 1 could form a seven membered ring with Gd(III), its donating ability is expected to be lower than a carboxylate due to increased electron density delocalization and charge neutrality. This does not however preclude weak binding of the carbamate moiety to the metal center in 1 giving a species that has one inner-sphere water molecule. The increased relaxivity of 1 compared to 2 is therefore most likely due to the increased mass of 1 (See, e.g., Caravan et al., Chem. Rev. 1999, 99, 2293-2352).

Due to enzyme instability in MOPS buffer, the relaxivities of 1 and 2 were determined in the enzyme kinetics buffer composed of phosphate and BSA (FIG. 9). Here, the results are higher in magnitude (4.73 vs. 3.68 mM⁻¹ sec⁻¹) but show the same trend as observed in MOPS buffer, namely a 20% drop in relaxivity upon going from 1 to 2. The 20% difference between the two agents may once again be simply the result of the increased mass of 1.

Although the relaxivity difference between 1 and 2 is similar in MOPS and phosphate/BSA buffer, the overall magnitude is markedly higher in the phosphate case. This trend would indicate that there is a bulk difference in the two buffers that is not specific to the nature of the individual contrast agent. As the relaxivity of small molecule Gd(III) chelates of a given mass is determined mainly by q and T_{R} (the rotational correlation time) (See, e.g., Merbach and Toth, The Chemistry of Contrast Agents in Medical Magnetic Resonance Imaging; John Wiley and Sons: West Sussex; N.Y., 2001; Caravan et al., Chem. Rev. 1999, 99, 2293-2352) it is conceivable that a difference in viscosity between the buffers could alter T_{R} and affect 1 and 2 in much the same manner. A comparison of the viscosities of the two solutions (See, e.g., Table 1) shows them to be nearly identical and the same

TABLE 1
Buffer Viscosity^{a}

| buffer | pH^{b} | viscosity^{b} (cP) |
|---|---|---|
| water | — | 0.692 ± 0.018 |
| acetate^{c} | 5 | 0.892 ± 0.030 |
| phosphate/BSA^{d} | 7.4 | 0.713 ± 0.018 |
| MOPS^{e} | 7.4 | 0.689 ± 0.005 |

^{a}determined by falling ball technique: for details see Experimental section. Data is the average of 5 runs ± 1 SD.
^{b}37° C.
^{c}100 mM sodium acetate.
^{d}100 mM sodium phosphate, 0.01% (w/v) bovine serum albumin (BSA).
^{e}10 mM MOPS, 100 mM NaCl.

as pure water. The presence of BSA may also confer some change in TR through non-specific binding. Targeting of serum albumin has been shown to yield a substantial (on the order of 10-fold) increase in relaxivity (Caravan et al., J. Am. Chem. Soc. 2002, 124, 3152-3162; Aime, et al., JBIC 1996, 1, 312-319; Doble et al., J. Am. Chem. Soc. 2001, 123, 10758-10759). Measurements in the absence of BSA however gave identical results (FIG. 9, phosphate columns). Thus, in some embodiments, the differences are due to specific interactions between the buffer salts and the contrast agents themselves with phosphate perhaps enhancing second-sphere relaxivity for both compounds.

The native lysosomal environment of β-glucuronidase is acidic with maximum activity observed between pH=4-5 (Himeno et al., J. Biochem. (Tokyo) 1974, 76, 1243-1252. The enzyme kinetics of 1 were examined (vide infra) and the relaxivity of compounds 1 and 2 were thus measured at pH=5.0. The results from two different buffers at the same pH are dramatically different. In 10 mM pyridine, 100 mM sodium chloride buffer, compound 1 has a relaxivity of 3.89±0.05 mM⁻¹ sec⁻¹ while 2 displays an r₁ of 4.16±0.09 mM⁻¹ sec⁻¹. In 100 mM acetate buffer, those values are 2.53±0.01 mM⁻¹ sec⁻¹ and 2.16±0.04 mM⁻¹ sec⁻¹ respectively. Within the same buffer, the agents are of approximately the same relaxivity, although the cleaved agent 2 has a higher relaxivity than 1 in pyridine buffer while it is lower in acetate.

No relaxivities have been reported in pyridine buffer making comparison difficult, but pyridine is expected to be a poor ligand for the oxophilic Gd(III) in water (e.g., a search of the CSD returned no structures containing a lanthanide coordinated to both a pyridyl and aquo ligand. The similarity in relaxivity between the two agents in pyridine buffer may be rationalized by partial dissociation of the aminoethyl arm in 2 upon protonation at this pH. This would increase the relaxivity of 2 rendering it comparable to the heavier, presumably q=1 compound 1. Conversely, the agents display half the observed relaxivity in acetate buffer as in pyridine and are low for q=1 complexes. This may be due, in some embodiments, to bidentate or multiple coordination of acetate with the Gd(III) ion in both 1 and 2 if the amine arm in 2 is protonated as postulated for the pyridine buffer. Inner-sphere water access would then be severely limited and the agents would generate relaxivity enhancement solely through outer and second sphere effects. At pH=7, propionate binding to GdDO3A has been shown to be monodentate (Aime et al., Chem. Comm. 2001, 115-116), and TbDO3A in the presence of a large excess of acetate gives a q value of one (Bruce et al., J. Am. Chem. Soc. 2000, 122, 9674-9684). The situation is more complex for the tricationic trisamide cyclen macrocyclic complexes. Here, methylation of the remaining macrocyclic nitrogen generates a complex with q=0.1 compared to 1.52 in the unmethylated species in the presence of carbonate (Bruce et al., J. Am. Chem. Soc. 2000, 122, 9674-9684). It is noted that these complexes have a much higher affinity for anions than 1 and 2 due to the increased cationic charge.

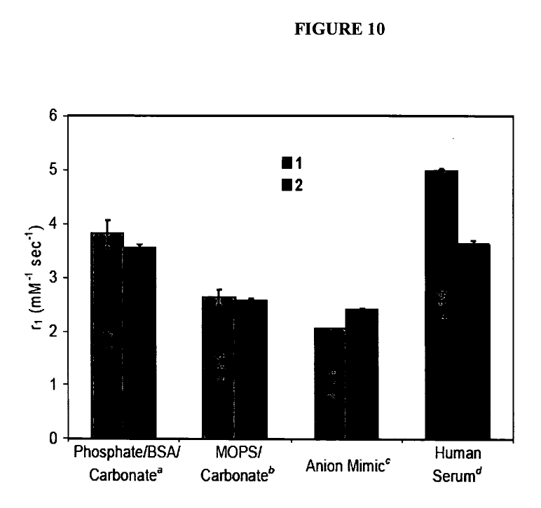

These studies on EGad and EGadMe demonstrate that in vitro measurements do not correlate with in vivo efficacy. While an MR contrast agent may display efficacy in vitro, the addition of all of the components found in blood plasma could result in a significant change in the properties of the agent. To investigate these effects and the interplay of coordinating bidentate anions on the present β-glucuronidase sensitive agent, the relaxivity of 1 and 2 were measured in buffers of increasing compositional complexity. These results are depicted in FIG. 10. For the MOPS and phosphate data, the effects are superimposed upon the overall relaxivity differences observed between the two buffers (FIG. 9). Addition of physiologically relevant carbonate concentrations (24 mM)³ to the buffers displayed in FIG. 9 gave similar results independent of buffer; namely the relaxivity of 1 decreased by 20-30% while that of 2 remained the same within error. This data supports the hypothesis of stronger binding of carbonate to 1 versus 2 and indicates that, in some embodiments, carbonate binding can displace the seven-membered carbamate chelate. The effect is not as prominent as desired since the addition of carbonate brings the relaxivity of the two agents to an equal value. Results with the anion extracellular mimic continue the trend begun in the carbonate-containing buffers. Here, the data exhibits the desired dark to bright (low to higher relaxivity) change. In this case it is a 17% increase. Once again, the decrease is more dramatic for 1 (22% decline) than 2 (6% drop), providing more evidence for the increased chelating anion affinity of 1 compared to 2.

If the coordination of endogenous anions was the sole intermolecular contributor to the relaxivities observed in these contrast agents, then the results from the anion extracellular mimic would translate well to the results developed in human blood serum. The dearth of literature on the relaxivity of Gd(III) contrast agents in human serum or plasma is remarkable given the ease and low cost of the experiment. For agents to be useful in vivo, they should display favorable characteristics in serum or plasma at the physiologically relevant temperature of 37° C. The present case shows the dramatic differences on going from a competitive extracellular anion mimic to human serum. The data is entirely different as compound 1 shows an increase in relaxivity of 240% while compound 2 displays a 150% increase. Furthermore, the relaxivity differential has switched with 1 27% brighter than 2 in serum. For these compounds in serum, the relaxivity indicates a species containing inner-sphere water molecules, in contrast to the q=0 species detected by Aime et al. for DO3A analogs in the presence of albumin (See, e.g., Aime et al., JBIC 2000, 5, 488-497). The complex composition of human serum makes it difficult to ascribe the results to any given component, but, it is contemplated that the higher viscosity and possible macromolecular interactions affect T_{R} and hence the relaxivity.

### Example 4

### Enzyme Kinetics

The use of a self-immolative linker that has demonstrated facile kinetics in chemotherapeutic prodrug applications was postulated to ameliorate the slow enzymatic cleavage rates observed for EGad and EGadMe (See, FIG. 3A). β-glucuronidase isolated from bovine liver was chosen for the current studies; this commercially available enzyme is more similar to the human variant than the E. coli version (See, e.g., Azoulay et al., Carbohydr. Res. 2001, 332, 151-156; Brot et al., Biochemistry 1978, 17, 385-391). Although the bacterial enzyme has much higher activity at extracellular pH (See, e.g., Jefferson et al., Proc. Natl. Acad. Sci. U.S.A. 1986, 83, 8447-8451), it is obviously not endogenous to humans and its use in ADEPT has resulted in host immune response (See, e.g., Pedley et al., Methods in Molecular Medicine 2004, 90, 491-514; Heine et al., Gene Therapy 2001, 8, 1005-1010), severely curtailing its potential. Furthermore, the 2-nitro-quinone-methide that results from enzymatic processing of 1 has been shown to not be an irreversible inhibitor of bovine β-glucuronidase (See, e.g., Azoulay et al., Carbohydr. Res. 2001, 332, 151-156). The large active site of β-glucuronidase and its homology to β-galactosidase indicated that the enzyme should tolerate the bulky macrocycle well (See, e.g., Jain et al., Nat. Struc. Bio. 1996, 3, 375-381).

Studies showed the enzyme to be stable for at least 2 hours at concentrations ≧0.5 mg/ml in phosphate buffer with 0.01% BSA at physiological conditions (pH=7.4, 37° C.). Below concentrations of 0.1 mg/ml stability was poor; as was stability in MOPS buffer. LC/MS provided a facile means to detect and identify the components of the kinetics experiments. At 2 h of reaction in the phosphate buffer described above, the presence of 4-hydroxy-3-nitrobenzyl alcohol could be easily detected in the LC data at 4.0 min, unreacted 1 was observed at 7.5 min and 2 appeared at 11.8 min. 1 had disappeared after 24 h.

The verification of the presence of 4-hydroxy-3-nitrobenzyl alcohol enabled the enzyme kinetics in phosphate/BSA buffer to be quantified using a continuous UV-visible assay. The alcohol has an absorption maximum at 422 nm, while 1 does not absorb at this wavelength. Initial rates were determined in phosphate/BSA buffer and phosphate/BSA/carbonate buffer (Table 2).

TABLE 2
Enzyine kinetic data for 1

| buffer | pH^{a} | initial rate^{a,b} | t_{1/2}^{c} |
|---|---|---|---|
| acetate^{d} | 5 | — | 19 ± 2 |
| phosphate/BSA^{e} | 7.4 | 158 ± 34 | — |
| phosphate/BSA/ | 7.4 | 148 ± 26 | — |
| carbonate^{f} | | | |

^{a}37° C.
^{b}0.2 mM 1, 1.0 mg/ml bovine liver β-glucuronidase; activity in nmol product/h/mg enzyme. Data average of 3 runs ± 1 S.D.
^{c}0.2 mM 1, 0.1 mg/ml bovine liver β-glucuronidase; 50% conversion time in minutes. Data average of 3 runs ±1 SD.
^{d}100 mM sodium acetate.
^{e}100 mM sodium phosphate, 0.01% (w/v) bovine serum albumin (BSA).
^{f}100 mM sodium phosphate, 0.01% (w/v) bovine serum albumin (BSA), 24 mM NaHCO₃.

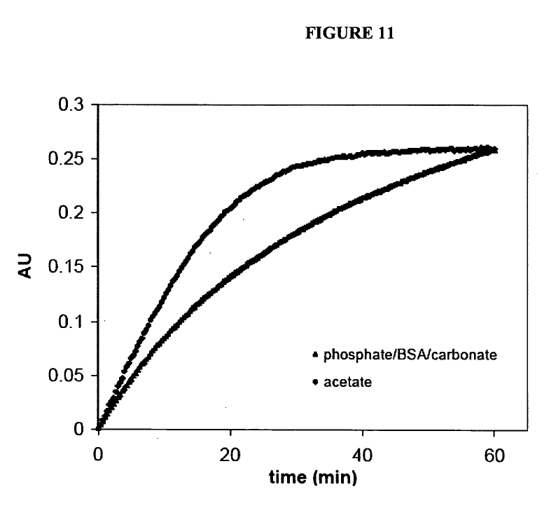

Representative kinetics trace are displayed in FIG. 11. Comparison with the initial rates determined using the standard fluorescent substrate 4-methylumbelliferyl-β-D-glucuronide at pH=5.5 (See, e.g., Tohyama et al., J. Biol. Chem. 2004, 279, 9777-9784) show the initial rates for 1 at pH=7.4 to be roughly 8% of the pH=5.5 data. These rates correlate well with the approximately 10% of native activity seen at pH=7.4 in the initial measurements of bovine β-glucuronidase activity (See, e.g., Himeno et al., J. Biochem. (Tokyo) 1974, 76, 1243-1252. These results are most likely due to the structural similarity near the glycosidic bond between 1 and the standard substrates and to the tolerance of the enzyme for variable substrates. Comparison of the kinetic data with that of the doxorubicin prodrug HMR 1826 is difficult since the prodrug kinetics were measured using the much more active E. coli enzyme (See, e.g., Florent et al., J. Med. Chem. 1998, 41, 3572-3581. The data in Table 2 show orders of magnitude improvement compared to in vitro EGad and EGadMe kinetics (See, e.g., Moats et al., Chem., Int. Ed. Engl. 1997, 36, 726-728; Louie et al., Nat. Biotechnol. 2000, 18, 321-325).

Kinetic measurements at the native enzyme pH of 5.0 are more difficult using the UV-visible assay since the 4-hydroxy-3-nitrobenzyl alcohol is protonated at this pH and hence its absorption is blue-shifted and overlaps that of 1. In addition, it is unknown whether the absorption change observed is due to 4-hydroxy-3-nitrobenzyl alcohol or the non-immolated linker-contrast agent conjugate. At pH=5, the phenol, whose pKₐ is about 7.5 should be protonated. For the self-immolation mechanism to occur the phenol must be deprotonated to allow the formation of the quinone methide. To verify immolation, the enzyme reaction was allowed to proceed to completion (no further change in absorption spectrum) and analyzed on LC using pH=5.0 buffer as eluent. These data show that immolation had occurred, but do not rule out the possibility that the immolation reaction occurred on the LC column. Kinetics determined by relaxivity (vide infra) indicate that 2 is formed during the enzymatic hydrolysis. Due to these complications, a half-life for 1 instead of an initial rate was determined and tabulated in Table 2 for pH=5 acetate buffer. The conversion was complete within 45 min at an enzyme concentration 10% of that used in the pH=7.4 measurements. This reflects the inherently faster kinetics at pH=5 and correlates well with the reported data at pH=5.

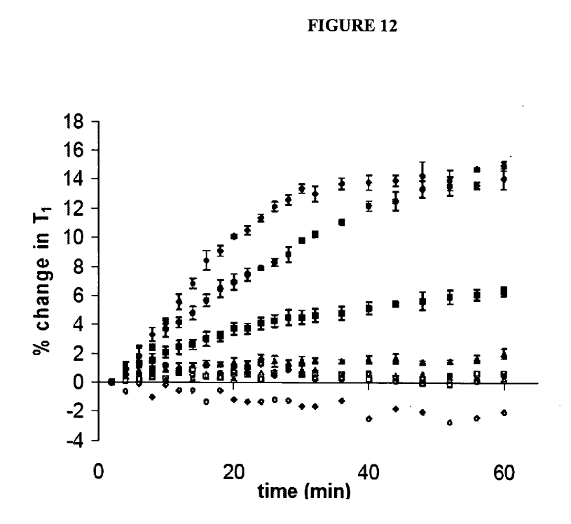

The kinetics can also be monitored using magnetic resonance. The time required to obtain accurate T₁ values using an inversion recovery pulse sequence were considerably longer than the kinetic processes under study, therefore a compromise was made between accuracy and resolution by using a saturation recovery sequence. In this fashion, the T₁ of the bulk water protons could be measured every 2 minutes to give reasonable T₁ estimates. The use of MR also allowed observation of kinetics in human serum, something that could not be done by visible light spectroscopy due to light scatter by suspended particles. FIG. 12 shows the normalized change in T₁ as a function of enzyme incubation time. The results show excellent correlation with the relaxivities of the substrate 1, and the product 2, measured in the absence of enzyme (See, e.g., FIGS. 9 and 10). The largest change detected for the pH=7.4 buffers comes from the data in human serum. Here, a 14% increase is observed over the course of one hour and the curve has not saturated at this time. The control without enzyme maintains a constant T₁ over this period. These serum experiments demonstrate that the contrast agent functions well in the complex biological milieu represented by human serum. Enzyme instability precluded determination at long reaction times, but the shapes of the curves match those obtained from the UV-visible assays, indicating that compound 2 forms on a time scale similar to the change in absorbance of the aromatic linker. In particular, the longitudinal relaxation time observed in acetate buffer levels off at +15% around 35 min corroborating well with both the complete conversion detected by absorption spectroscopy (See Table 2 and FIG. 11) and the 15% decrease in relaxivity observed between the pure compounds in acetate buffer.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising a compound for use as a contrast agent in magnetic resonance imaging, said compound comprising: a sensor component and a macrocycle containing an MRI agent, wherein the contrast agent is configured to decompose and release said MRI agent in the presence of a glucuronidase.

2. The composition of claim 1, wherein said sensor component comprises beta-glucuronic acid.

3. The composition of claim 1, wherein said compound further comprises a linker that attaches said sensor to said macrocycle.

4. The composition of claim 1, wherein said MRI agent comprises a gadolinium (III) ion.

5. The composition of claim 1, wherein said glucuronidase comprises a beta-glucuronidase.

6. A kit comprising the composition of claim 1.

7. A method for imaging a tissue, comprising:
a) exposing a tissue to a contrast agent comprising a sensor component and an MRI agent, wherein said contrast agent is configured to decompose and release said MRI agent in the presence of a glucuronidase; and
b) imaging said tissue via magnetic resonance imaging.

8. The method of claim 7, wherein said tissue comprises necrotic tumor tissue.

9. The method of claim 7, wherein said tissue is located in vivo in a subject.

10. The method of claim 9, wherein said subject is a human.

11. The method of claim 7, wherein said sensor component comprises beta-glucuronic acid.

12. The method of claim 7, wherein said contrast agent further comprises a linker that attaches said sensor to a macrocycle containing said MRI agent.

13. The method of claim 7, wherein said MRI agent comprises a gadolinium (III) ion.

14. The method of claim 7, wherein said glucuronidase comprises a beta-glucuronidase.

15. The method of claim 12, wherein said contrast agent comprises a glycosidic bond that is hydrolyzed in the presence of said glucuronidase to cause decomposition of said linker, releasing said MRI agent.
